(19) United States
(12) Patent Application Publication
(10) Pub. No.: US 2017/0240652 A1
(43) Pub. Date: Aug. 24, 2017

(54) ANTIBODIES AGAINST SEROTONIN, TRYPTOPHAN AND KYNURENINE METABOLITES AND USES THEREOF

(71) Applicant: Nestec S.A., Vevey (CH)

(72) Inventors: Fabiyola Selvaraj, San Diego, CA (US); Fred Princen, La Jolla, CA (US); Sharat Singh, Rancho Santa Fe, CA (US)

(73) Assignee: Nestec S.A., Vevey (CH)

(21) Appl. No.: 15/587,739

(22) Filed: May 5, 2017

Related U.S. Application Data
(63) Continuation of application No. PCT/IB2015/058976, filed on Nov. 19, 2015.
(60) Provisional application No. 62/082,047, filed on Nov. 19, 2014.

(51) Int. Cl. C07K 16/44

(52) U.S. Cl. CPC C07K 16/44; C07K 2317/33

(57) ABSTRACT

The present invention provides antibodies and methods for preparing antibodies to metabolites in the serotonin, tryptophan and kynurenine pathways, such as 5-hydroxyindole-3-acetic acid (5-HIAA), melatonin (MT) and kynurenic acid (KYNA). The specific metabolite antibodies have low cross-reactivity to structurally related metabolites, and are useful reagents for specific and sensitive immunoassays. The present invention also provides methods for using such antibodies to measure the levels of 5-HIAA, melatonin, or kynurenic acid in biological samples from human patients.

### CROSS-REFERENCES TO RELATED APPLICATIONS

The present application is a continuation of PCT/IB2015/058976 filed on Nov. 19, 2015, which claims priority to U.S. Provisional Application No. 62/082,047, filed Nov. 19, 2014, the contents of which are incorporated by reference in its entirety for all purposes.

### BACKGROUND OF THE INVENTION

Irritable bowel syndrome (IBS) is the most common of all gastrointestinal disorders, affecting 10-20% of the general population and accounting for more than 50% of all patients with digestive complaints. However, studies suggest that only about 10% to 50% of those afflicted with IBS actually seek medical attention. Patients with IBS present with disparate symptoms such as, for example, abdominal pain predominantly related to defecation, diarrhea, constipation or alternating diarrhea and constipation, abdominal distention, gas, and excessive mucus in the stool. More than 40% of IBS patients have symptoms so severe that they have to take time off from work, curtail their social life, avoid sexual intercourse, cancel appointments, stop traveling, take medication, and even stay confined to their house for fear of embarrassment. The estimated health care cost of IBS in the United States is $8 billion per year (Talley et al., Gastroenterol., 109:1736-1741 (1995)).

IBS patients are classified into three groups according to their predominant bowel symptoms: constipation-predominant IBS (IBS-C), diarrhea-predominant IBS (IBS-D), IBS with alternating symptoms of diarrhea and constipation (IBS-M), and unsubtyped IBS (IBS-U). In current clinical practice, diagnosis of IBS is based on the Rome III criteria and according to the symptoms presented by the patients. There are no specific biological, radiographic, endoscopic or physiological biomarkers that can be used to identify this disorder.

Irritable bowel syndrome is a heterogeneous gastrointestinal (GI) function disorder. There is increasing evidence pointing to the involvement of stress response and the immune system in its pathogenesis. Stress, e.g., acute or chronic stress, can impact on almost all aspects of the gut including gastrointestinal motility, visceral perception, gastrointestinal secretion, intestinal permeability, and the intestinal microbiota. IBS is often described as a disorder of the brain-gut axis. Serotonin (5-HT) is an important neurotransmitter and signaling molecule in the central nervous system (CNS) and the enteric nervous system (ENS) of the brain-gut axis. Serotonin is generated in the CNS and the ENS by the conversion of tryptophan, an essential amino acid. About 95% of the body's total serotonin is found in the gut (Kesztheylyi et al., 2015, Neurogastroenterol Motil, 27(8):1127-1137) Tryptophan is converted to 5-hydroxytryptophan (5-HTP) by tryptophan hydroxylase, and 5-HTP is converted to serotonin by aromatic amino acid decarboxylase. Tryptophan can also be metabolized along the kynurenine pathway to generate neurotoxic and neuroprotective metabolites by enzymes that are either immuno-responsive or stress-responsive (Kennedy et al., World J Gastoenterol, 20(39): 14105-14125).

The precise pathophysiology of IBS remains to be elucidated. It has been proposed that melatonin, a metabolite of serotonin, has strong anti-oxidant and anti-inflammatory activity, and can regulate intestinal motility (Konturek et al., J Physiol Pharmacol, 2007, 58:381-405; Siah et al., World J Gastroenterol, 2014, 20(10):2492-2498). It also appears to have an inhibitory effect on smooth muscle motor activity (Bebeuik and Pang, J Pineal Res, 1994, 16:91-99). Studies have shown that levels of melatonin are reduced in the gut in post-menopausal women with IBS-C (Chojnacki et al., Endokrynol Pol, 2013, 64(2):114-20).

Kynurenic acid (KYNA) is another metabolite of the tryptophan, serotonin, and kynurenine pathways that may contribute of IBS. 1% of ingested tryptophan is converted into serotonin, while the majority is catabolized via the kynurenine pathway. Patients with IBS can have decreased levels of mucosal KYNA which may contribute to functional, neural, metabolic or inflammatory changes that facilitate the development of IBS (Keszthelyi et al., J Psycho Res, 2013, 74:5001-504). In the intestines, KYNA has neuroprotective, anti-oxidative and anti-inflammatory properties and may have a role in gut motility and sensory functions.

Therapeutics drugs directed to the serotonin pathway are currently under investigation for the treatment of IBS. Treatment with melatonin may alleviate bowel pain associated with IBS-C (Elsenbruch, Gut, 2005, 54(10):1353-1354) and may improve abdominal pain in IBS-D patients with a sleep disturbance (Song et al., Gut, 2005, 54:1402-1407).

In view of the foregoing, there is a need in the art for methods of measuring or quantitating the levels of metabolites of the tryptophan, serotonin and kynurenine pathways in a biological sample from a subject. In addition, methods are need for diagnosing IBS in an individual by monitoring the brain-gut-microbiome axis. Assays are needed to assess whether various metabolic and catabolic pathways are functioning properly. The present invention satisfies these and other needs.

### BRIEF SUMMARY OF THE INVENTION

In one aspect, provided herein is an isolated antibody or antibody fragment thereof that specifically binds to 5-hydroxyindole-3-acetic acid (5-HIAA) and has less than 1% cross-reactivity to one or more members selected from the group consisting of tryptophan (Trp), serotonin (5-HT), 5-hydroxytryptophan (5-HTP), kynurenine (KYN), kynurenic acid (KYNA), 3-hydroxykynurenine (3-HK), 3-hydroxyanthranilic acid (3-HAA), quinolinic acid (QUIN), anthranilic acid (ANA), serotonin-O-sulfate, serotonin-O-phosphate, and melatonin (MT). The isolated antibody or purified antibody can be a polyclonal antibody or a monoclonal antibody. In some embodiments, the isolated antibody or purified antibody is a chimeric or a humanized antibody. The isolated or purified antibody fragment thereof can be a Fab fragment, a Fab′ fragment or F(ab)′₂ fragment.

In some embodiments, the antibody or antibody fragment thereof against 5-HIAA is produced by the hybridoma cell line deposited on Nov. 17, 2015 under ATCC Accession Number PTA-122671 and designated 1204-10G6F11H3.

In one embodiment, the antibody or antibody fragment thereof is produced by immunizing an animal with an immunogen comprising a 5-HIAA derivative conjugated to a carrier protein under conditions such that immune cells of the animal produce an antibody or antibody fragment thereof that specifically binds to 5-HIAA; and isolating the antibody or antibody fragment thereof from the animal. The animal can be a goat, rabbit or mouse. In some embodiments, the 5-HIAA derivative comprises a benzoxazole derivative of 5-HIAA.

In another aspect, provided herein is an isolated antibody or antibody fragment thereof that specifically binds to melatonin (MT) and has less than 1% cross-reactivity to one or more members selected from the group consisting of tryptophan (Trp), serotonin (5-HT), 5-hydroxytryptophan (5-HTP), 5-hydroxyindole-3-acetic acid (5-HIAA), kynurenine (KYN), kynurenic acid (KYNA), 3-hydroxykynurenine (3-HK), 3-hydroxyanthranilic acid (3-HAA), quinolinic acid (QUIN), anthranilic acid (ANA), serotonin-O-sulfate, and serotonin-O-phosphate. The isolated antibody or purified antibody can be a polyclonal antibody or a monoclonal antibody. In some embodiments, the isolated antibody or purified antibody is a chimeric or a humanized antibody. The isolated or purified antibody fragment thereof can be a Fab fragment, a Fab′ fragment or F(ab)′₂ fragment.

In some embodiments, the antibody or antibody fragment thereof against melatonin is produced by the hybridoma cell line deposited on Nov. 17, 2015 under ATCC Accession Number PTA-122669 and designated 1212-6C1E2F7.

In one embodiment, the antibody or antibody fragment thereof is produced by immunizing an animal with an immunogen comprising melatonin conjugated to a carrier protein under conditions such that immune cells of the animal produce an antibody or antibody fragment thereof that specifically binds to melatonin; and isolating the antibody or antibody fragment thereof from the animal. The animal can be a goat, rabbit or mouse.

In yet another aspect, provided herein is an isolated antibody or antibody fragment thereof that specifically binds to kynurenic acid (KYNA), which has less than 1% cross-reactivity to one or more members selected from the group consisting of tryptophan (Trp), serotonin (5-HT), 5-hydroxytryptophan (5-HTP), 5-hydroxyindole-3-acetic acid (5-HIAA), kynurenine (KYN), 3-hydroxykynurenine (3-HK), 3-hydroxyanthranilic acid (3-HAA), quinolinic acid (QUIN), anthranilic acid (ANA), serotonin-O-sulfate, serotonin-O-phosphate, and melatonin (MT). The isolated antibody or purified antibody can be a polyclonal antibody or a monoclonal antibody. In some embodiments, the isolated antibody or purified antibody is a chimeric or a humanized antibody. The isolated or purified antibody fragment thereof can be a Fab fragment, a Fab′ fragment or F(ab)′₂ fragment.

In some embodiments, the antibody or antibody fragment thereof against kynurenic acid is produced by the hybridoma cell line deposited on Nov. 17, 2015 under ATCC Accession Number PTA-122670 and designated 1194-6H5B11A7.

In one embodiment, the antibody or antibody fragment thereof is produced by immunizing an animal with an immunogen comprising kynurenic acid (KYNA) conjugated to a carrier protein under conditions such that immune cells of the animal produce an antibody or antibody fragment thereof that specifically binds to KYNA; and isolating the antibody or antibody fragment thereof from the animal. The animal can be a goat, rabbit or mouse.

In some embodiments, any one of the isolated antibodies or antibody fragments thereof described herein has a detectable label.

In some embodiments, any one of the isolated antibodies or antibody fragments thereof described herein is immobilized on a solid substrate.

In one aspect, provided herein is a hybridoma cell line that produces and secretes monoclonal antibodies which selectively bind to 5-HIAA and that has been deposited under ATCC Accession No. PTA-122671 on Nov. 17, 2015 and designated 1204-10G6F11H3.

In some aspects, provided herein is a hybridoma cell line that produces and secretes monoclonal antibodies which selectively bind to melatonin and that has been deposited under ATCC Accession No. PTA-122669 on Nov. 17, 2015 and designated 1212-6C1E2F7.

In one aspect, provided herein is a hybridoma cell line that produces and secretes monoclonal antibodies which selectively bind to kynurenic acid and that has been deposited under ATCC Accession No. PTA-122670 on Nov. 17, 2015 and designated 1194-6H5B11A7.

Also provided herein is a method for detecting the level of 5-HIAA in a sample from a patient suspected of having irritable bowel syndrome using an immunoassay. The method comprises: (a) contacting the isolated antibody or antibody fragment thereof described above, a sample obtained from the patient, and immobilized 5-HIAA under suitable conditions to form a complex comprising the isolated antibody or antibody fragment thereof and 5-HIAA present in the sample or the immobilized 5-HIAA; (b) detecting the level of antibody or antibody fragment thereof bound to the complex comprising the immobilized 5-HIAA; and (c) calculating the level of 5-HIAA in the sample based on the level of antibody or antibody fragment thereof from step (b).

In some embodiments, the isolated antibody or antibody fragment thereof, the sample, and the immobilized 5-HIAA are contacted simultaneously. In other embodiments, the isolated antibody or antibody fragment thereof, the sample, and the immobilized 5-HIAA are contacted sequentially. The immunoassay can be an ELISA such as a competitive ELISA.

In one aspect, provided herein is a method for detecting the level of melatonin in a sample from a patient suspected of having irritable bowel syndrome using an immunoassay. The method comprises: (a) contacting the isolated antibody or antibody fragment thereof described above, a sample obtained from the patient, and immobilized melatonin under suitable conditions to form a complex comprising the isolated antibody or antibody fragment thereof and melatonin present in the sample or the immobilized melatonin; (b) detecting the level of antibody or antibody fragment thereof bound to the complex comprising the immobilized melatonin; and (c) calculating the level of melatonin in the sample based on the level of antibody or antibody fragment thereof from step (b).

In some embodiments, the isolated antibody or antibody fragment thereof, the sample, and the immobilized melatonin are contacted simultaneously. In other embodiments, the isolated antibody or antibody fragment thereof, the sample, and the immobilized melatonin are contacted sequentially. The immunoassay can be a competitive ELISA.

In one aspect, provided herein is a method for detecting the level of kynurenic acid (KYNA) in a sample from a patient suspected of having irritable bowel syndrome using an immunoassay. The method comprises: (a) contacting the isolated antibody or antibody fragment thereof described herein, a sample obtained from the patient, and immobilized KYNA under suitable conditions to form a complex comprising the isolated antibody or antibody fragment thereof and KYNA present in the sample or the immobilized KYNA; (b) detecting the level of antibody or antibody fragment thereof bound to the complex comprising the immobilized KYNA; and (c) calculating the level of KYNA in the sample based on the level of antibody or antibody fragment thereof from step (b).

In some embodiments, the isolated antibody or antibody fragment thereof, the sample, and the immobilized kynurenic acid are contacted simultaneously. In other embodiments, the isolated antibody or antibody fragment thereof, the sample, and the immobilized kynurenic acid are contacted sequentially. The immunoassay can be a competitive ELISA.

This application incorporates by reference International Patent Application Publication Nos. WO2014/188377 and WO2014/188378 in their entirety for all purposes.

These and other aspects, advantages and embodiments will become more apparent when read with the detailed description and figures which follow.

### BRIEF DESCRIPTION OF THE DRAWINGS

FIG. 1 illustrates metabolites of the serotonin, tryptophan and kynurenine pathways. Metabolites include tryptophan (Trp, 122), 5-hydroxytryptophan (5-HTP, 125), serotonin (5-HT, 101), melatonin (MT, 120), 5-hydroxyindole acetic acid (5-HIAA or 5HIAA, 115), kynurenine (KYN, 131), kynurenic acid (KYNA, 135), anthranilic acid (ANA, 140), 3-hydroxykynurenine (3-HK, 146), 3-hydroxyanthranilic acid (3-HAA, 149), quinolinic acid (QUIN; 160), and xanthurenic acid (XA, 148).

FIG. 2 illustrates an exemplary embodiment of a competitive ELISA described herein.

FIGS. 3A-3D show immunogenic conjugates used to generate antibodies described herein. The immunogens include benzoxazole derivative of 5-HIAA (FIG. 3A), melatonin (FIG. 3B), and kynurenic acid (FIG. 3C) haptens. The haptens were conjugated to a carrier protein via a linker such as a PEG linker. FIG. 3D provides a HPLC chromatogram showing the separation of derivatized metabolites includes derivatized serotonin (5HT-d), and derivatized 5-hydroxyindole acetic acid (5-HIAA-d).

FIGS. 4A and 4B provide schematic diagrams of antibody production using immunogenic conjugates. FIG. 4A shows that the immunogenic conjugates can be used for monoclonal antibody production and polyclonal antibody production. FIG. 4B illustrates the process of monoclonal antibody production including immunization of a mouse with a designated antigen, generation of hybridoma clones, and isolation of monoclonal antibodies specific to the designated antigen.

FIGS. 5A and 5B provide chemical schemes for synthetically generating 5-HIAA derivatives. FIG. 5A shows a benzoxazole derivative of 5-HIAA conjugated to a PEG linker. FIG. 5B shows a benzoxazole derivative of 5-HIAA conjugated to biotin via a PEG linker.

FIGS. 6A-6D show the reactivity of mouse monoclonal antibodies produced by the hybridoma clone 10G6F11H3. The antibodies specifically binds (are immunoreactive) to 5-HIAA. FIG. 6A shows that undiluted antibody and a 1:200 dilution of the monoclonal antibody bind 5-HIAA similarly. FIG. 6B shows data from a competitive assay between free 5-HIAA and immobilized 5-HIAA. A higher OD was detected when no free 5-HIAA (0 ng/mL) was added to the well compared to when 100 ng/mL of free 5-HIAA was present. A high OD corresponds to a high level of antibody bound to immobilized 5-HIAA. A low OD corresponds to a low level of antibody bound to immobilized 5-HIAA, and a high level of antibody bound to free 5-HIAA in this assay. FIG. 6C shows the titration of the monoclonal antibody at various dilutions at different concentrations of free 5-HIAA. FIG. 6D shows that the monoclonal antibody against 5-HIAA has no cross-reactivity to serotonin and the monoclonal antibody against 5HT has no cross-reactivity to 5-HIAA.

FIGS. 7A and 7B show that monoclonal antibodies from the hybridoma clone 10G6F11H3 have specificity for 5-HIAA, but not for tryptophan, serotonin or kynurenine metabolites. FIG. 7A shows that the antibodies have no cross-reactivity to 4-hydroxyquinoline, 3-hydroxy-DL-kynurenine and melatonin. FIG. 7B shows no cross-reactivity to serotonin (5-HT), 5-hydroxy-L-tryptophan, and N-acetyl-5-hydroxytryptamine. The monoclonal antibody generated from hybridoma clone 10G6F11H3 is an anti-5HIAA is an IgG₁κ antibody.

FIG. 8 shows a standard curve for the monoclonal antibody against 5-HIAA. The concentrations ranged from 0 ng/mL to 100 ng/mL with a dilution factor of 5.

FIG. 9 shows the presence of polyclonal antibodies against melatonin in antisera from rabbits #16401, #16402, and #16403 at pre-bleed and bleeds 1-9 (B1, B2, B3, B4, B5, B6, B7, B8 and B9). The rabbits were immunized with the melatonin immunogenic conjugate described herein. Rabbit #16401 exhibited the highest titer of anti-melatonin antibody.

FIGS. 10A and 10B show the reactivity of affinity purified rabbit polyclonal anti-melatonin antibodies in a competitive ELISA. In the assay, melatonin (25 μg/mL) was immobilized onto the surface of the well. Free (not immobilized or unbound) melatonin and affinity purified rabbit polyclonal anti-melatonin antibodies were added to the wells. The amount of free melatonin that was added ranged from 0.00 mM (right of graph) to 8.00 mM (left of graph). The OD measurement represents the amount of antibody bound to the immobilized melatonin. In a similar competitive assay, other competing (free, not immobilized or unbound) compounds that are structurally similar to melatonin were incubated with the antibodies and immobilized melatonin. FIG. 10B shows that the affinity purified rabbit antibody has no cross-reactivity to serotonin (Ser), tryptophan (Tryp), or 5-HIAA.

FIG. 11 illustrates the specificity of monoclonal antibodies against melatonin. The graph shows that antibodies from 4 hybridoma clones (6C1E2F7, 6C2H4C8, 7C7F1G2, and 7C8A1D2) specifically bind to melatonin and lacks cross-reactivity to serotonin, tryptophan, and 5-HIAA. The monoclonal antibody generated from hybridoma clone 6C1E2F7 is an anti-melatonin IgG₃κ antibody.

FIG. 12 provides a standard curve for the monoclonal anti-melatonin antibody from hybridoma clone 6C1E2F7.

FIGS. 13A and 13B show that the reactivity of rabbit polyclonal antibodies against kynurenic acid (KYNA). FIG. 13A illustrates that antiserum from a rabbit immunized with the KYNA immunogenic conjugate described herein contains antibodies that specifically bind to KYNA. FIG. 13A shows results from a competitive ELISA assay where free KYNA competes with immobilized KYNA for antibody binding. In this assay the amount of free KYNA ranged from 0 μg/mL (right) to 500 μg/mL (left). The OD measurement represents the amount of antibody bound to the immobilized KYNA. When no free KYNA was added (0.00 μg/mL), the anti-KYNA antibody bound to the immobilized KYNA antigen, as represented by the high OD value. When free KYNA antigen was added, less antibody was bound to the immobilized antigen, as represented by the lower OD value. FIG. 13B shows results from a similar competitive assay. In this assay the amount of antibody was also varied from a dilution of 1:250 to a dilution of 1:2500.

FIGS. 14A and 14B show the reactivity of mouse monoclonal antibodies to kynurenic acid (KYNA). FIG. 14A shows that antibodies from hybridoma clones 4B11H9A2 and 6H5B11A7 specifically bind to KYNA and have no cross-reactivity to 3-OH-DL-kynurenine, serotonin, tryptophan, N-acetyl-5-hydroxy-tryptamine, and 5-OH-quinoline. In the competitive ELISA, compounds that are structurally similar to KYNA did not interfere with the binding of the antibody to KYNA. FIG. 14B shows that undiluted and diluted mouse monoclonal anti-KYNA antibodies bind to KYNA. The monoclonal antibody generated from hybridoma clone 6H5B11A7 is an anti-KYNA is an IgG₁κ antibody.

FIGS. 15A and 15B show that the mouse monoclonal antibodies produced by hybridoma clone 6H5B11A7 specifically bind to kynurenic acid. As shown in FIG. 15A, free KYNA antigen competes with immobilized KYNA antigen for antibody binding in the competitive ELISA provided herein. With increasing amounts of free KYNA, less antibody binds to the immobilized antigen and the OD value decreases. FIG. 15B shows a standard curve for the mouse monoclonal anti-KYNA antibody.

FIGS. 16A and 16B show the results from an exemplary embodiment of the competitive ELISA disclosed herein. In FIG. 16A, a TMB substrate was used for the colorimetric reaction. In FIG. 16B, a luminescent substrate was used for the detection reaction. The assay using the luminescent substrate provides more sensitivity than the TMB substrate assay.

### DETAILED DESCRIPTION OF THE INVENTION

### I. Definitions

The terms “a,” “an,” or “the” as used herein not only includes aspects with one member, but also includes aspects with more than one member. For example, an embodiment including “a polyamine compound and an excipient” should be understood to present certain aspects with at least a second polyamine compound, at least a second excipient, or both.

The term “antigen” refers to any molecule, compound, composition, or particle that can bind specifically to an antibody. An antigen has one or more epitopes that interact with the antibody, although it does not necessarily induce production of that antibody.

The term “antibody” refers to an immunoglobulin molecule that is immunologically reactive with a particular antigen, and includes both polyclonal and monoclonal antibodies. The term also includes genetically engineered forms such as chimeric antibodies (e.g., humanized murine antibodies) and heteroconjugate antibodies (e.g., bispecific antibodies). The term “antibody” also includes antigen binding forms of antibodies, including fragments with antigen-binding capability e.g., Fab′, F(ab′)₂, Fab, Fv, scFv and di-scFv (see, e.g., Kuby, Immunology, 3^{rd} Ed., W. H. Freeman & Co., New York 1998). The term further includes bivalent or bispecific molecules, diabodies, triabodies, and tetrabodies. Bivalent and bispecific molecules are described in, e.g., Zhu et al. (Protein Sci. 1997; 6:781-9, and Hu et al. (Cancer Res. 1996; 56:3055-61). While various antibody fragments are defined in terms of the digestion of an intact antibody, one of skill will appreciate that fragments can be synthesized de novo either chemically or by utilizing recombinant DNA methodology. Thus, the term antibody, as used herein also includes antibody fragments either produced by the modification of whole antibodies or synthesized using recombinant DNA methodologies

An antibody can consist of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. An “antibody” functions as a binding protein and is structurally defined as comprising an amino acid sequence from or derived from the framework region of an immunoglobulin encoding gene of an animal producing antibodies.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies can include V_{H}-V_{L} dimers, including single chain antibodies (antibodies that exist as a single polypeptide chain), such as single chain Fv antibodies (sFv or scFv) in which a variable heavy and a variable light region are joined together (directly or through a peptide linker) to form a continuous polypeptide. The single chain Fv antibody is a covalently linked V_{H}-V_{L} which may be expressed from a nucleic acid including V_{H}- and V_{L}-encoding sequences either joined directly or joined by a peptide-encoding linker (e.g., Huston, et al. Proc. Nat. Acad. Sci. USA, 85:5879-5883, 1988). While the V_{H} and V_{L} are connected to each as a single polypeptide chain, the V_{H} and V_{L} domains associate non-covalently. Alternatively, the antibody can be another fragment. Other fragments can also be generated, e.g., using recombinant techniques, as soluble proteins or as fragments obtained from display methods. Antibodies can also include diantibodies and miniantibodies. Antibodies of the invention also include heavy chain dimers, such as antibodies from camelids. Thus, in some embodiments an antibody is dimeric. In other embodiments, the antibody may be in a monomeric form that has an active isotype. In some embodiments the antibody is in a multivalent form, e.g., a trivalent or tetravalent form, that can cross-link the antigen.

The term “antibody fragment” or “antigen binding fragment” refers to at least a portion of the variable region of the immunoglobulin molecule, which binds to its target, i.e., the antigen recognition domain or the antigen binding region. Some of the constant region of the immunoglobulin may be included. Examples of antibody fragments include, but are not limited to, linear antibodies, single-chain antibody molecules (scFv), Fv fragments, hypervariable regions to complementarity determining regions (CDRs), VL (light chain variable region), VH (heavy chain variable region), Fab fragments, F(ab)′₂ fragments, multispecific antibodies formed from antibody fragments, and any combination of those or any other portion of an immunoglobulin peptide capable of binding to target antigen. As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology.

The term “polyclonal antibody” refers to an antibody within a collection of antibodies secreted by different B cell lineages that recognize multiple epitopes on the same antigen.

The term “monoclonal antibody” refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site or epitope. Furthermore, in contrast to polyclonal antibody preparations which typically include different antibodies directed against different determinants or epitopes, each monoclonal antibody is directed against a single determinant on the antigen. Monoclonal antibodies to be used in accordance with the present invention may be made by the hybridoma method first described by Kohler and Milstein, Nature, 256:495 (1975), or may be made by recombinant DNA methods such as described in U.S. Pat. No. 4,816,567. In some cases, monoclonal antibodies may also be isolated from phage libraries generated using the techniques described in McCafferty et al., Nature, 348:552-554 (1990).

The term “chimeric antibody” refers to an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region, or portion thereof, having a different or altered antigen specificity; or with corresponding sequences from another species or from another antibody class or subclass.

The term “humanized antibody” refers to an antibody in which the antigen binding loops, i.e., complementarity determining regions (CDRs), comprised by the VH and VL regions are grafted to a human framework sequence. Typically, the humanized antibodies have the same binding specificity as the non-humanized antibodies described herein. Techniques for humanizing antibodies are well known in the art and are described in e.g., Verhoyen et al., Science, 239: 1534 (1988) and Winter and Milstein, Nature, 349: 293 (1991).

The phrase “specifically (or selectively) binds” to an antibody or “specifically (or selectively) immunoreactive with,” when referring to an antigen or hapten, refers to a binding reaction that is determinative of the presence of the antigen or hapten, often in a heterogeneous population of antigens or haptens and other biologics such as a mixture of cells, a cell lysate or a biological sample, e.g., blood, plasma or serum. Thus, under designated immunoassay conditions, the specified antibodies bind to a particular antigen or hapten (at least two times the background and more typically more than 10 to 100 times background. Specific binding to an antibody under such conditions requires an antibody that is selected for its specificity for a particular antigen or hapten. For example, polyclonal antibodies can be selected to obtain only those polyclonal antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

Specific binding can be measured, for example, by methods known in the art, e.g., using competition assays with a control molecule that is similar to the target, for example, an excess of non-labeled target. An antibody that specifically binds a target antigen can have a K_{d} for the antigen of at least about 10⁻⁴ M, alternatively at least about 10⁻⁵ M, alternatively at least about 10⁻⁶ M, alternatively at least about 10⁻⁷ M, alternatively at least about 10⁻⁸ M, alternatively at least about 10⁻⁹ M, alternatively at least about 10⁻¹⁰ M, alternatively at least about 10⁻¹¹ M, alternatively at least about 10⁻¹² M, or greater. In one embodiment, the term “specific binding” refers to binding where an antibody binds to its particular hapten without substantially binding to any other structurally similar haptens or compounds. In such embodiments, the extent of non-specific binding is the amount of binding at or below background and will typically be less than about 10%, preferably less than about 5%, and more preferably less than about 1% as determined by fluorescence activated cell sorting (FACS) analysis, enzyme-linked immunosorbent assay (ELISA) or radioimmunoprecipitation (MA), for example.

The term “cross-reactivity” refers to the relative binding of a designated (primary) antigen and a secondary antigen to a purified antibody of interest, wherein the designated or primary antigen is used to produce the antibody of interest. C50_{secondary} is the concentration of the secondary antigen required to cause 50% inhibition of the reaction between the primary antigen and the antibody of interest. Similarly, C50_{primary} is the concentration of the primary antigen required to cause 50% inhibition of the reaction between the primary antigen and the antibody (self-inhibition). Then the relative equilibrium binding constant for the variant antigen, C50_{primary}/C50_{secondary}, measures cross-reactivity (Benjamin and Perdue, Methods, 1996, 9(3):508-515). In other words, the percent cross-reactivity of an antibody produced against compound X with respect to a specific compound X is equal to [(a/b)×100] where a is the amount of compound X required to displace 50% of compound Y bound of the antibody; b is the amount of compound Y required to displace 50% of compound X bound to the antibody. The term “cross-reactivity” of an antibody can also refer to the interaction of an antibody to similar or dissimilar epitopes on different antigens. “Cross-reactivity” can be measured using standard assays known to one skilled in the art, such as a competitive ELISA, e.g., a direct competitive ELISA or an indirect competitive ELISA.

As used herein, the term “isolated” or “purified” antibody refers to an antibody that is substantially or essentially free from components that normally or naturally accompany it. Purity and homogeneity are typically determined using analytical chemistry techniques such as polyacrylamide gel electrophoresis or high performance liquid chromatography. Contaminant components of its environment are materials that would interfere with uses for the antibody or fragment thereof, and may include enzymes, hormones, and other proteinaceous or non-proteinaceous solutes. In certain embodiments, the isolated antibody is purified to greater than 95% by weight of polypeptides as determined by the Lowry method, and preferably, more than 99% by weight, or to homogeneity by SDS-page under reducing or non-reducing conditions using Coomassie blue, or silver stain. An isolated antibody includes the antibody in situ within recombinant cells. In some cases, an isolated antibody is prepared by a least one purification step.

The term “hybridoma cell line” or “hybridoma clone” refers to a hybrid cell line used to produce a monoclonal antibody. In some cases, a hybridoma cell is an antibody-producing cell from a mouse's spleen fused to a myeloma cell, wherein the mouse has been injected with a specific antigen.

The term “hapten” refers to a small molecule that can elicit an immune response in an animal when the hapten is linked or conjugated to a carrier molecule, e.g., a carrier protein, to form an immunogen or an immunogenic conjugate. The hapten-carrier protein complex is immunogenic (can elicit an immune response) and the hapten alone (unbound hapten) is not immunogenic. Non-limiting examples of a carrier protein include bovine serum albumin (BSA), mouse serum albumin (MSA), rabbit serum albumin (RSA), ovalbumin (OVA), keyhole limpet hemocyanin (KLH), bovine or porcine thyroglobulin, tetanus toxoid, gelatin, or soybean trypsin inhibitor and the like.

The term “immunogen” refers to a substance, compound, peptide, or composition which stimulates the production of an immune response in an animal.

As used herein, a “linker” or “spacer” is any molecule capable of binding (e.g., covalently) together a hapten to another molecule or moiety disclosed herein. Linkers include, but are not limited to, straight or branched chain carbon linkers, heterocyclic carbon linkers, peptide linkers, polyether linkers and short hydrophilic molecules. Exemplary linkers can include but are not limited to NH—CH₂—CH₂—O—CH₂—CO— and 5-amino-3-oxopentanoyl. For example, poly(ethylene glycol) linkers are available from Quanta Biodesign, Powell, Ohio. These linkers optionally have amide linkages, sulfhydryl linkages, or hetero functional linkages.

The term “label” or a “detectable label” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or peptides and proteins which can be made detectable, e.g., by incorporating a radiolabel into a peptide. The detectable label can be, without limitation, a fluorescent label, a luminescent label, a chemiluminescent label, a bioluminescent label, a radioactive label or an enzymatic label.

The term “solid substrate” or “solid support” refers to a solid material, membrane, array, chip, bead, and the like. The surface on the solid substrate can be composed of the same material as the substrate. The surface may be composed of any of a wide variety of materials, for example, polymers, plastics, resins, polysaccharides, silica or silica-based materials, carbon, metals, inorganic glasses, membranes, or any of the above-listed substrate materials.

The term “immunoassay” refers to an assay that detects or measures the presence or concentration (level or amount) of an analyte (small molecule, chemical compound, peptide, polypeptide, biomolecule, antigen, metabolite, etc.) by utilizing an antibody, immunoglobulin or a fragment thereof.

The terms “subject,” “patient,” and “individual” are used interchangeably and refer to except where indicated, mammals such as humans and non-human primates, as well as rabbits, rats, mice, goats, pigs, and other mammalian species.

The term “sample” includes any biological specimen obtained from an individual. Suitable samples for use include, without limitation, whole blood, plasma, serum, saliva, urine, stool, tears, any other bodily fluid, tissue samples (e.g., biopsy), and cellular extracts thereof (e.g., red blood cellular extract). In a preferred embodiment, the sample is a serum or plasma sample. The use of samples such as serum, saliva, and urine is well known in the art (see, e.g., Hashida et al., J. Clin. Lab. Anal., 11:267-86 (1997)). One skilled in the art will appreciate that samples such as serum samples can be diluted prior to performing the methods disclosed herein.

“Acyl” as used herein includes an alkanoyl, aroyl, heterocycloyl, or heteroaroyl group as defined herein. Representative acyl groups include acetyl, benzoyl, nicotinoyl, and the like.

“Alkanoyl” as used herein includes an alkyl-C(O)- group wherein the alkyl group is as defined herein. Representative alkanoyl groups include acetyl, ethanoyl, and the like.

“Alkenyl” as used herein includes a straight or branched aliphatic hydrocarbon group of 2 to about 15 carbon atoms that contains at least one carbon-carbon double or triple bond. Preferred alkenyl groups have 2 to about 12 carbon atoms. More preferred alkenyl groups contain 2 to about 6 carbon atoms. In one aspect, hydrocarbon groups that contain a carbon-carbon double bond are preferred. In a second aspect, hydrocarbon groups that contain a carbon-carbon triple bond are preferred (i.e., alkynyl). “Lower alkenyl” as used herein includes alkenyl of 2 to about 6 carbon atoms. Representative alkenyl groups include vinyl, allyl, n-butenyl, 2-butenyl, 3-methylbutenyl, n-pentenyl, heptenyl, octenyl, decenyl, propynyl, 2-butynyl, 3-methylbutynyl, n-pentynyl, heptynyl, and the like.

An alkenyl group can be unsubstituted or optionally substituted. When optionally substituted, one or more hydrogen atoms of the alkenyl group (e.g., from 1 to 4, from 1 to 2, or 1) may be replaced with a moiety independently selected from the group consisting of fluoro, hydroxy, alkoxy, amino, alkylamino, acylamino, thio, and alkylthio.

“Alkenylene” as used herein includes a straight or branched bivalent hydrocarbon chain containing at least one carbon-carbon double or triple bond. Preferred alkenylene groups include from 2 to about 12 carbons in the chain, and more preferred alkenylene groups include from 2 to 6 carbons in the chain. In one aspect, hydrocarbon groups that contain a carbon-carbon double bond are preferred. In a second aspect, hydrocarbon groups that contain a carbon-carbon triple bond are preferred. Representative alkenylene groups include —CH═CH—, —CH₂—CH═CH—, —C(CH₃)═CH—, —CH₂CH═CHCH₂—, ethynylene, propynylene, n-butynylene, and the like.

“Alkoxy” as used herein includes an alkyl-O— group wherein the alkyl group is as defined herein. Representative alkoxy groups include methoxy, ethoxy, n-propoxy, i-propoxy, n-butoxy, heptoxy, and the like.

An alkoxy group can be unsubstituted or optionally substituted. When optionally substituted, one or more hydrogen atoms of the alkoxy group (e.g., from 1 to 4, from 1 to 2, or 1) may be replaced with a moiety independently selected from the group consisting of fluoro, hydroxy, alkoxy, amino, alkylamino, acylamino, thio, and alkylthio.

“Alkoxyalkyl” as used herein includes an alkyl-O-alkylene- group wherein alkyl and alkylene are as defined herein. Representative alkoxyalkyl groups include methoxyethyl, ethoxymethyl, n-butoxymethyl and cyclopentylmethyloxyethyl.

“Alkoxycarbonyl” as used herein includes an ester group; i.e., an alkyl-O—CO— group wherein alkyl is as defined herein. Representative alkoxycarbonyl groups include methoxycarbonyl, ethoxycarbonyl, t-butyloxycarbonyl, and the like.

“Alkoxycarbonylalkyl” as used herein includes an alkyl-O—CO-alkylene- group wherein alkyl and alkylene are as defined herein. Representative alkoxycarbonylalkyl include methoxycarbonylmethyl, ethoxycarbonylmethyl, methoxycarbonylethyl, and the like.

“Alkyl” as used herein includes an aliphatic hydrocarbon group, which may be straight or branched-chain, having about 1 to about 20 carbon atoms in the chain. Preferred alkyl groups have 1 to about 12 carbon atoms in the chain. More preferred alkyl groups have 1 to 6 carbon atoms in the chain. “Branched-chain” as used herein includes that one or more lower alkyl groups such as methyl, ethyl or propyl are attached to a linear alkyl chain. “Lower alkyl” as used herein includes 1 to about 6 carbon atoms, preferably 5 or 6 carbon atoms in the chain, which may be straight or branched. Representative alkyl groups include methyl, ethyl, n-propyl, isopropyl, n-butyl, t-butyl, n-pentyl, and 3-pentyl.

An alkyl group can be unsubstituted or optionally substituted. When optionally substituted, one or more hydrogen atoms of the alkyl group (e.g., from 1 to 4, from 1 to 2, or 1) may be replaced with a moiety independently selected from the group consisting of fluoro, hydroxy, alkoxy, amino, alkylamino, acylamino, thio, and alkylthio.

“Alkylene” as used herein includes a straight or branched bivalent hydrocarbon chain of 1 to about 6 carbon atoms. Preferred alkylene groups are the lower alkylene groups having 1 to about 4 carbon atoms. Representative alkylene groups include methylene, ethylene, and the like.

“Alkylthio” as used herein includes an alkyl-S— group wherein the alkyl group is as defined herein. Preferred alkylthio groups are those wherein the alkyl group is lower alkyl. Representative alkylthio groups include methylthio, ethylthio, isopropylthio, heptylthio, and the like.

“Alkylthioalkyl” as used herein includes an alkylthio-alkylene- group wherein alkylthio and alkylene are defined herein. Representative alkylthioalkyl groups include methylthiomethyl, ethylthiopropyl, isopropylthioethyl, and the like.

“Amido” as used herein includes a group of formula Y₁Y₂N—C(O)— wherein Y₁ and Y₂ are independently hydrogen, alkyl, or alkenyl; or Y₁ and Y₂, together with the nitrogen through which Y₁ and Y₂ are linked, join to form a 4- to 7-membered azaheterocyclyl group (e.g., piperidinyl). Representative amido groups include primary amido (H₂N—C(O)—), methylamido, dimethylamido, diethylamido, and the like. Preferably, “amido” is an —C(O)NRR′ group where R and R′ are members independently selected from the group consisting of H and alkyl. More preferably, at least one of R and R′ is H.

“Amidoalkyl” as used herein includes an amido-alkylene- group wherein amido and alkylene are defined herein. Representative amidoalkyl groups include amidomethyl, amidoethylene, dimethylamidomethyl, and the like.

“Amino” as used herein includes a group of formula Y₁Y₂N— wherein Y₁ and Y₂ are independently hydrogen, acyl, or alkyl; or Y₁ and Y₂, together with the nitrogen through which Y₁ and Y₂ are linked, join to form a 4- to 7-membered azaheterocyclyl group (e.g., piperidinyl). Optionally, when Y₁ and Y₂ are independently hydrogen or alkyl, an additional substituent can be added to the nitrogen, making a quaternary ammonium ion. Representative amino groups include primary amino (H₂N—), methylamino, dimethylamino, diethylamino, and the like. Preferably, “amino” is an —NRR′ group where R and R′ are members independently selected from the group consisting of H and alkyl. Preferably, at least one of R and R′ is H.

“Aminoalkyl” as used herein includes an amino-alkylene- group wherein amino and alkylene are defined herein. Representative aminoalkyl groups include aminomethyl, aminoethyl, dimethylaminomethyl, and the like.

“Aroyl” as used herein includes an aryl-CO— group wherein aryl is defined herein. Representative aroyl include benzoyl, naphth-1-oyl and naphth-2-oyl.

“Aryl” as used herein includes an aromatic monocyclic or multicyclic ring system of 6 to about 14 carbon atoms, preferably of 6 to about 10 carbon atoms. Representative aryl groups include phenyl and naphthyl.

“Aromatic ring” as used herein includes 5-12 membered aromatic monocyclic or fused polycyclic moieties that may include from zero to four heteroatoms selected from the group consisting of oxygen, sulfur, selenium, and nitrogen. Exemplary aromatic rings include benzene, pyrrole, furan, thiophene, imidazole, oxazole, thiazole, triazole, pyrazole, pyridine, pyrazine, pyridazine, pyrimidine, naphthalene, benzathiazoline, benzothiophene, benzofurans, indole, benzindole, quinoline, and the like. The aromatic ring group can be substituted at one or more positions with halo, alkyl, alkoxy, alkoxy carbonyl, haloalkyl, cyano, sulfonato, amino sulfonyl, aryl, sulfonyl, aminocarbonyl, carboxy, acylamino, alkyl sulfonyl, amino and substituted or unsubstituted substituents.

“Biomolecule” as used herein includes a natural or synthetic molecule for use in biological systems. Preferred biomolecules include a protein, a peptide, an enzyme substrate, a hormone, an antibody, an antigen, a hapten, an avidin, a streptavidin, a carbohydrate, a carbohydrate derivative, an oligosaccharide, a polysaccharide, and a nucleic acid. More preferred biomolecules include a protein, a peptide, an avidin, a streptavidin, or biotin.

“Carboxy” and “carboxyl” as used herein include a HOC(O)— group (i.e., a carboxylic acid) or a salt thereof.

“Carboxyalkyl” as used herein includes a HOC(O)-alkylene- group wherein alkylene is defined herein. Representative carboxyalkyls include carboxymethyl (i.e., HOC(O)CH₂—) and carboxyethyl (i.e., HOC(O)CH₂CH₂—).

“Cycloalkyl” as used herein includes a non-aromatic mono- or multicyclic ring system of about 3 to about 10 carbon atoms, preferably of about 5 to about 10 carbon atoms. More preferred cycloalkyl rings contain 5 or 6 ring atoms. A cycloalkyl group optionally comprises at least one sp²-hybridized carbon (e.g., a ring incorporating an endocyclic or exocyclic olefin). Representative monocyclic cycloalkyl groups include cyclopentyl, cyclohexyl, cyclohexenyl, cycloheptyl, and the like. Representative multicyclic cycloalkyl include 1-decalin, norbornyl, adamantyl, and the like.

“Cycloalkylene” as used herein includes a bivalent cycloalkyl having about 4 to about 8 carbon atoms. Preferred cycloalkylenyl groups include 1,2-, 1,3-, or 1,4- cis- or trans-cyclohexylene.

“Halo” or “halogen” as used herein includes fluoro, chloro, bromo, or iodo.

“Heteroatom” as used herein includes an atom other than carbon or hydrogen. Representative heteroatoms include O, S, and N. The nitrogen or sulphur atom of the heteroatom is optionally oxidized to the corresponding N-oxide, S-oxide (sulfoxide), or S,S-dioxide (sulfone). In a preferred aspect, a heteroatom has at least two bonds to alkylene carbon atoms (e.g., —C₁-C₉ alkylene-O—C₁-C₉ alkylene-). In some embodiments, a heteroatom is further substituted with an acyl, alkyl, aryl, cycloalkyl, heterocyclyl, or heteroaryl group (e.g., —N(Me)-; —N(Ac)—).

“Hydroxyalkyl” as used herein includes an alkyl group as defined herein substituted with one or more hydroxy groups. Preferred hydroxyalkyls contain lower alkyl. Representative hydroxyalkyl groups include hydroxymethyl and 2-hydroxyethyl.

“Linking group” i.e., L, comprises the atoms joining the metabolite derivative with a biomolecule such as a carrier protein, a biotin or streptavidin. See also R. Haugland, Molecular Probes Handbook of Fluorescent Probes and Research Chemicals, Molecular Probes, Inc. (1992). In one embodiment, L represents the linking group precursor before the attachment reaction with a protein, and R¹¹ represents the resultant attachment between the compound of the invention and the protein or biotin (i.e., R¹¹ is the resultant attachment between the linking group joined to the biomolecule). Preferred reactive functionalities include phosphoramidite groups, an activated ester (e.g., an NHS ester), thiocyanate, isothiocyanate, maleimide and iodoacetamide. L may comprise a terminal amino, carboxylic acid, or sulfhydryl group covalently attached to the ring. In certain instances, the terminal amino, carboxylic acid, or sulfhydryl group is shown and is represented as -L-NH₂, or -L-C(O)OH or -L-SH.

“Oxo” as used herein includes a group of formula >C═O (i.e., a carbonyl group —C(O)—).

“Sulfonato” as used herein includes an —SO₃⁻ group, preferably balanced by a cation such as H⁺, Na⁺, K⁺, and the like.

“Sulfonatoalkyl” as used herein includes a sulfonato-alkylene- group wherein sulfonato and alkylene are as defined herein. A more preferred embodiment includes alkylene groups having from 2 to 6 carbon atoms, and a most preferred embodiment includes alkylene groups having 2, 3, or 4 carbons. Representative sulfonatoalkyls include sulfonatomethyl, 3-sulfonatopropyl, 4-sulfonatobutyl, 5-sulfonatopentyl, 6-sulfonatohexyl, and the like.

### II. Detailed Description of Embodiments

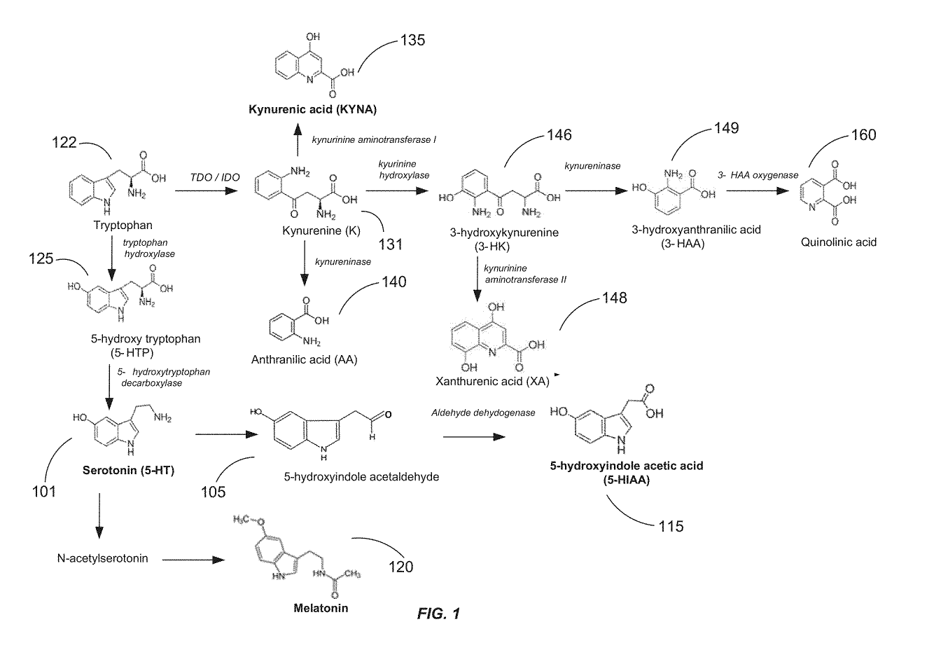

In certain aspects, the present disclosure provides assays, e.g., immunoassays for measuring the level, amount or concentration of a metabolite of the tryptophan, serotonin and kynurenine pathways in a sample obtained from a subject, e.g., a human subject. For example, with reference to FIG. 1, provided herein are compositions and methods for measuring or quantitating the amount of 5-HIAA (5-hydroxyindole-3-acetic acid) 115, melatonin, and kynurenic acid in a biological sample, e.g., blood, plasma, or serum obtained from a subject suspected of having or having irritable bowel syndrome (IBS). Provided herein are antibodies, e.g., polyclonal and monoclonal antibodies that are immunoreactive to specific tryptophan, serotonin and kynurenine pathway metabolites. As such, the compositions and methods can be used to aid in the diagnosis or prognosis of IBS or other pathological conditions involving the tryptophan, serotonin and kynurenine pathways, such as carcinoid syndrome, depression, hypertension, autism Alzheimer's and migraine.

Prior art methods of detecting or measuring metabolites that are structurally similar are either nonexistent or lack sensitivity, specificity and reproducibility. Generally, the methods are unable to distinguish between structurally similar compounds. With some methods, sample volumes of about 500 μL are required to measure the level of specific metabolites. Also, in some cases, the sample must undergo processing such as extraction, lyophilization and/or reconstitution prior to performing the method.

One of ordinary skill in the art recognizes that serotonin and 5-HIAA are sensitive to oxygen and very unstable. Degradation of these compounds occurs at 4° C. in about 7 hours from thawing. The unstable nature of the 5-hydroxyindoles can lead to unreliable assay measurements, even when additives are used to prevent oxidative breakdown.

### A. Tryptophan and Serotonin Pathway Metabolites—5-HIAA Haptens

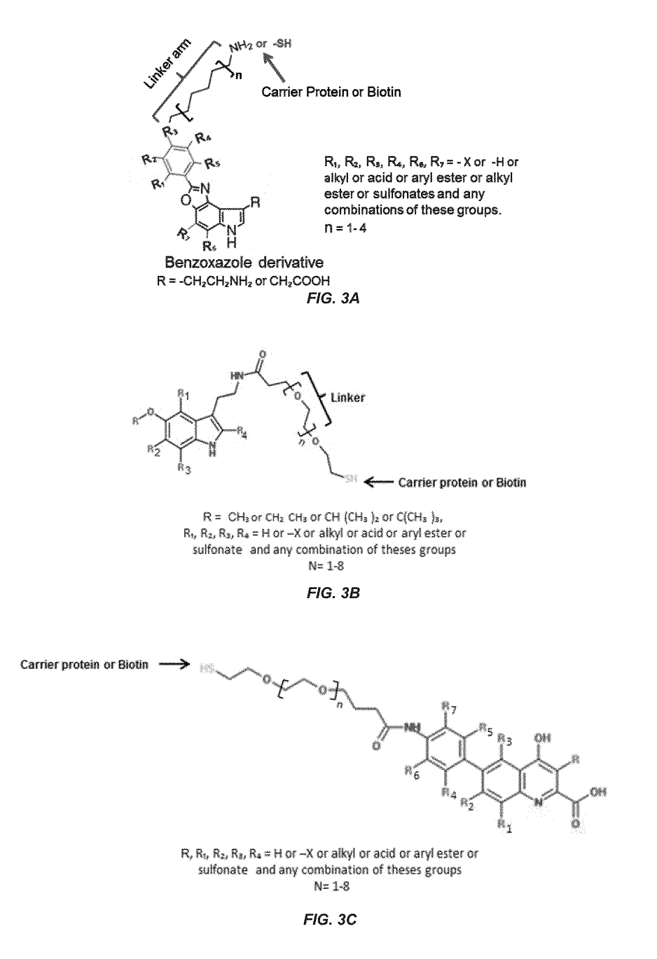

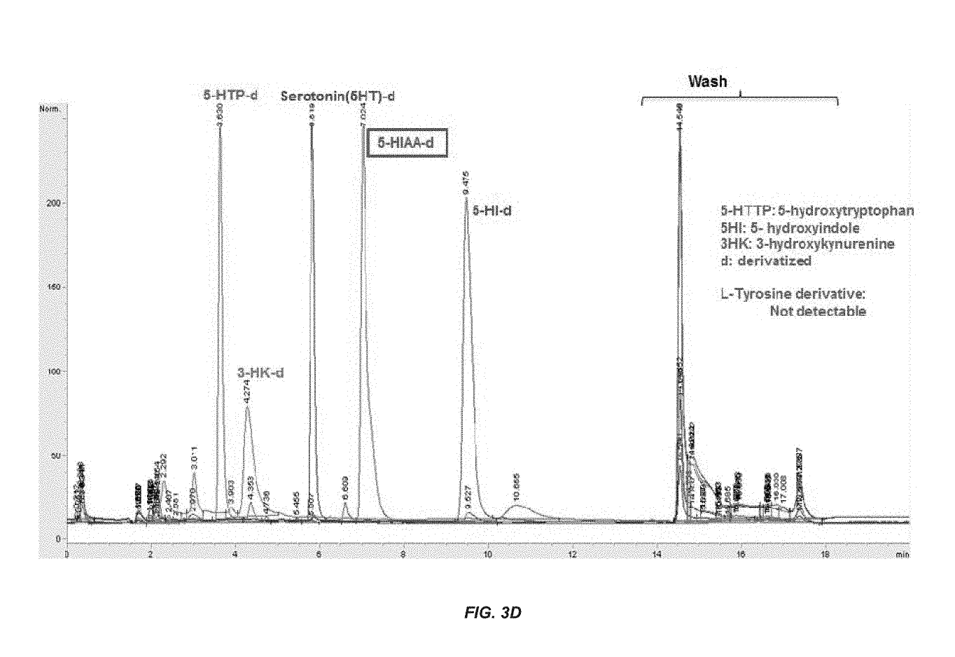

In one aspect, the present invention provides metabolite derivatives and conjugates thereof, methods for antibody production and antibodies of serotonin metabolites. In certain aspects, derivatization is preferred as metabolites such as 5-HT and 5-HIAA are sensitive to oxygen, and thus unstable. The level of serotonin in plasma can range from about 0.6 to 179 nmol/L. Chemical derivatization of 5-HT and 5-HIAA under mild conditions stabilizes the compounds. Thus, in one aspect, the present invention provides stable benzoxazole derivatives of serotonin metabolites. The stable benzoxazole derivatives can be detected by HPLC with high sensitivity due to their fluorescence (FIG. 3D). The derivative can be conjugated to a biomolecule such as a carrier protein and combined with an adjuvant to stimulate an immune response. The derivative can also be conjugated to other biomolecules including peptides.

The present invention provides stable derivatives of serotonin (5-HT) and 5-hydroxyindole acetic acid (5-HIAA). In one aspect, the present invention provides a compound of Formula I:

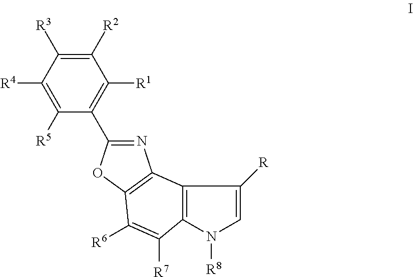

wherein R is a member selected from the group consisting of alkyl, alkoxy, alkoxyalkyl, aminoalkyl, amidoalkyl, carboxyalkyl, substituted carboxyalkyl; and

R¹, R², R³, R⁴, R⁵, R⁶, R⁷ and R⁸ are each a member independently selected from the group consisting of hydrogen, alkyl, halo, hydroxyl, alkoxy, amino, aroyl, alkanoyl, amido, substituted amido, cyano, carboxyl, alkoxycarbonyl, sulfonato, alkoxyalkyl, carboxy, carboxyalkyl, alkoxycarbonylalkyl, sulfonatoalkyl, L and R¹¹B;

L is a linker;

R¹¹ is the resultant attachment between the compound and a biomolecule; and

B is a biomolecule.

In one aspect, R is a member selected from the group consisting of aminoalkyl, carboxyalkyl, and substituted carboxyalkyl. In another aspect, R is a member selected from the group consisting of —CH₂CH₂NH₂, —CH₂CH₂CO₂H and —CH₂CH(NH₂)CO₂H.

L represents a linking group for attachment to a biomolecule such as a carrier protein or biotin. In some embodiments, L comprises polyethylene glycol or PEG. For example, L may comprise a terminal amino, carboxylic acid, or sulfhydryl group covalently attached to the ring. In certain instances, the terminal amino, carboxylic acid, or sulfhydryl group is shown and is represented as -L-NH₂, or -L-C(O)OH or -L-SH.

R¹¹ represents the resultant attachment between the compound of the invention and a biomolecule such as a carrier protein, a peptide or biotin (i.e., R¹¹ comprises the linking group joined to a biomolecule).

L is a member selected from the group consisting of a direct link, or a covalent linkage, wherein the covalent linkage is linear or branched, cyclic or heterocyclic, saturated or unsaturated, having 1-60 atoms selected from the group consisting of C, N, P, O, and S, wherein L can have additional hydrogen atoms to fill valences, wherein the linkage contains any combination of ether, thioether, amine, ester, carbamate, urea, thiourea, oxy or amide bonds; or single, double, triple or aromatic carbon-carbon bonds; or phosphorus-oxygen, phosphorus-sulfur, nitrogen-nitrogen, nitrogen-oxygen, or nitrogen-platinum bonds; or aromatic or heteroaromatic bonds. In certain aspects, L comprises a terminal amino, carboxylic acid, or sulfhydryl group.

In certain aspects, L is of the formula:

—X¹—Y¹—X²—

wherein: X¹ is a member selected from the group of a bivalent radical, a direct link, oxygen, an optionally substituted nitrogen and sulfur; Y¹ is a member selected from the group of a direct link and C₁-C₁₀ alkylene optionally interrupted by a heteroatom; and X² is a member selected from the group of a bivalent radical, a direct link, oxygen, an optionally substituted nitrogen and sulfur.

Preferably, the bivalent radical of X¹ and X² are each independently selected from the group of a direct link, optionally substituted alkylene, optionally substituted alkyleneoxycarbonyl, optionally substituted alkylenecarbamoyl, optionally substituted alkylenesulfonyl, arylenesulfonyl, optionally substituted aryleneoxycarbonyl, optionally substituted arylenecarbamoyl, optionally substituted thiocarbonyl, a optionally substituted sulfonyl, and optionally substituted sulfinyl.

In certain preferred aspects, L is —(CH₂)ₙ—, wherein r is an integer from 1 to 10, preferably n is an integer from 1 to 5, such as 1 to 4, or 1, 2, 3, 4, or 5.

In addition, the benzoxazole derivatives can be used to make immunogenic conjugates. For example, in one aspect, the conjugates of the present invention are used to raise an immunogenic response that is specific to the metabolite of interest. In certain instances, a benzoxazole derivative and a linker arm (wherein n is about 1-20) can be used to append a carrier protein to an amino (or sulfhydryl) end. In some embodiments, the linker arm is PEG. The linker arm may include a PEG₁, PEG₂, PEG₃, PEG₄, PEG₅, PEG₆, PEG₇, PEG₈, PEG₉, PEG₁₀, PEG₁₁, PEG₁₂, PEG₁₃, PEG₁₄, PEG₁₅, PEG-₁₆, PEG-₁₇, PEG-₁₈, PEG-₁₉, or PEG20 linker. In some embodiments, the 5-HIAA derivative hapten is described herein.

To test the affinity and specificity of the antibody thus produced, a biotinylated hapten can be made. In certain instances, a benzoxazole derivative and a linker arm (wherein n is about 1-20) can be used to append a biotin molecule to an amino (or sulfhydryl) end. In some embodiments, the linker arm is PEG. The linker arm may include a PEG₁, PEG₂, PEG₃, PEG₄, PEG₅, PEG₆, PEG₇, PEG₈, PEG₉, PEG₁₀, PEG₁₁, PEG₁₂, PEG₁₃, PEG₁₄, PEG₁₅, PEG₁₆, PEG₁₇, PEG₁₈, PEG₁₉, or PEG₂₀ linker. In some embodiments, the biotin molecule is substituted for different molecule that can be used to immobilize the hapten to a solid substrate or support.

In some embodiments, the benzoxazole derivative is an oxazolo-indole-PEG-biotin-ester or an oxazolo-indole-PEG-biotin-acid.

In certain aspects, a compound of the serotonin pathway, as shown in FIG. 1 or a compound of Formula I, can be reacted with a carrier molecule using conjugation chemistry well known in the art. For example, an activated ester (an NHS ester) can react with a primary amine to make a stable amide bond. A maleimide and a thiol can react together and make a thioether. Alkyl halides react with amines and thiols to make alkylamines and thioethers, respectively. Any derivative providing a reactive moiety that can be conjugated to a protein can be utilized herein. As is known in the art, moieties comprising a free amino group, a free carboxylic acid group, or a free sulfhydryl group provide useful reactive groups for protein conjugation. For example, a free amino group can be conjugated to proteins via glutaraldehyde cross-linking, or via carbodiimide cross-linking to available carboxy moieties on the protein. Also, a hapten with a free sulfhydryl group can be conjugated to proteins via maleimide activation of the protein, e.g., using sulfosuccinimidyl-4-(N-maleimidomethyl)cyclohexane-1-carboxylate (Sulfo-SMCC), then linkage to the sulfhydryl group.

When linking a carrier protein having a carboxylic acid group for attachment to an amine containing metabolite, the carboxylic acid can first be converted to a more reactive form using an activating reagent, to form for example, a N-hydroxy succinimide (NHS) ester or a mixed anhydride. The amine-containing metabolite is treated with the resulting activated acid to form an amide linkage. One of skill in the art will recognize that alternatively, the NHS ester can be on the metabolite and the amine can be on the carrier protein.

The process of stabilizing the metabolite by derivatization allows for generation of antibodies to the immunogenic conjugate. With the antibodies in hand, an immunoassay such as ELISA can be used wherein the antibody is highly specific to the metabolite of interest.

As is illustrated in FIG. 1, metabolites of interest in the serotonin pathway are for example, serotonin (5-HT) 101, 5-hydroxyindole acetaldehyde 105 and 5-hydroxyindole acetic acid (5-HIAA) 115. In one aspect, the present invention provide an isolated or purified antibody or antigen binding fragment thereof that specifically binds to 5-hydroxyindole acetic acid (5-HIAA) 115, wherein the antibody has less than 1% cross-reactivity, e.g., 0.9%, 0.8%, 0.7%, 0.6%, 0.5%, 0.4%, 0.3%, 0.2%, 0.1% or 0% cross-reactivity to one or more metabolites selected from the group consisting of tryptophan 122, 5-hydroxytryptophan 125, serotonin 101, melatonin 120, kynurenine 131, kynurenic acid 135, anthranilic acid 140, 3-hydroxykynurenine 146, 3-hydroxyanthranilic acid 149, quinolinic acid 160, and xanthurenic acid 148 of FIG. 1.

In one aspect, the present invention provides isolated or purified antibodies to metabolite conjugates. Firstly, a metabolite or stable derivative thereof can be prepared. Next, a carrier protein such as BSA is conjugated to the derivative. Antibodies against the metabolite or stable derivative thereof were made by injecting the conjugate into a mammal such as a rabbit, mouse, sheep, chicken, goat and the like. Thereafter, the biotinylated haptan can be used to test the reactivity, binding activity, specificity, and/or sensitivity of the antibody so produced.

In other aspects, the present invention provides methods for making antibodies, e.g., polyclonal antibodies or monoclonal antibodies that specifically bind to a serotonin metabolite. The method comprises: (a) providing an immunogen comprising a derivative selected from the group consisting of serotonin (5-HT), 5-hydroxyindole acetic acid (5-HIAA), and 5-hydroxy tryptophan (5-HTP) each derivative conjugated to a carrier protein; (b) immunizing an animal with the immunogen under conditions such that the immune system of the animal makes the antibodies; and(c) removing the antibodies from the animal.

In one aspect, the antibody generated according to the method provided herein can be removed or isolated from serum or cell culture supernatant using the conjugates of the present invention. For example, in one aspect, a 5-HIAA compound, a conjugate thereof or a derivative thereof can be used to remove the antibody from the serum of an immunized animal, such as an immunized goat, rabbit or mouse. The antibody can be purified by selectively enriching or specifically isolating antibodies of interest from serum, ascites fluid, cell culture supernatant or media, and the like. For example, an affinity method such as an antigen-specific affinity method or an immunoglobulin class-specific affinity method can be used to isolate antibodies of interest. The biotinylated 5-HIAA compound can be used to remove their corresponding antibodies from a mammal (such as a rabbit, mouse or goat).

In some aspect, the present invention provides an isolated or purifed monoclonal antibody that is immunoreactive to 5-HIAA and is produced by a hybridoma cell line deposited at American Type Culture Collection (ATCC) on Nov. 17, 2015 under ATCC Accession No. PTA-122671 and designated 1204-10G6F11H3. Such an antibody has substantially no cross-reactivity to other structurally similar metabolites or compounds of the tryptophan, serotonin, and kynurenine pathway including tryptophan 122, 5-hydroxytryptophan 125, serotonin 101, melatonin 120, kynurenine 131, kynurenic acid 135, anthranilic acid 140, 3-hydroxykynurenine 146, 3-hydroxyanthranilic acid 149, quinolinic acid 160, and xanthurenic acid 148 of FIG. 1.

### B. Tryptophan and Serotonin Pathway Metabolites—Melatonin Haptens

Provided herein is a stable melatonin hapten, a variant thereof, or a derivative thereof that can be conjugated to a biomolecule such as a carrier protein and combined with an adjuvant to stimulate an immune response.

In another aspect, the present invention provides antigens for antibody production of metabolites in the tryptophan pathway. In certain instance, irregularities of serotonin function in irritable bowel syndrome (MS) are due to changes in the metabolism of the a serotonin metabolite, i.e., melatonin 120 (FIG. 1).

The present invention provides antibodies and methods for preparing antibodies to melatonin (MT).

In one aspect, the present invention provides a derivative of melatonin having the structure of Formula II:

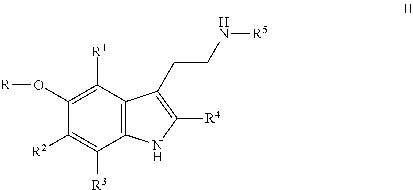

R is selected from the group consisting of hydrogen, alkyl, aroyl, alkanoyl, amido, substituted amido, L and R¹¹B;

R¹, R², R³, R⁴ and R⁵ are each a member independently selected from the group consisting of hydrogen, alkyl, halo, carboxyl, hydroxyl, alkoxy, aroyl, alkanoyl, amido, substituted amido, alkoxycarbonyl, sulfonato, alkoxyalkyl, carboxyalkyl, alkoxycarbonylalkyl, sulfonatoalkyl, L and R¹¹B;

L is a linker;

R¹¹ is the resultant attachment between the compound and a biomolecule; and

B is a biomolecule.

In another aspect, the compound of Formula II can be used to conjugate a carrier protein using conjugation chemistry well known in the art in order to make antibodies. An activated ester (an NHS ester) can react with a primary amine to make a stable amide bond. A maleimide and a thiol can react together and make a thioether. Alkyl halides react with amines and thiols to make alkylamines and thioethers, respectively. Any derivative providing a reactive moiety that can be conjugated to a protein can be utilized herein. As is known in the art, moieties comprising a free amino group, a free carboxylic acid group, or a free sulfhydryl group provide useful reactive groups for protein conjugation. For example, a free amino group can be conjugated to proteins via glutaraldehyde cross-linking, or via carbodiimide cross-linking to available carboxy moieties on the protein. Also, a hapten with a free sulfhydryl group can be conjugated to proteins via maleimide activation of the protein, e.g., using sulfosuccinimidyl-4-(N-maleimidomethyl)cyclohexane-1-carboxylate (Sulfo-SMCC), then linkage to the sulfhydryl group.

An exemplary schematic for one conjugation is as follows, wherein n is an integer from 0 to 20 (1, 2, 3, 4, 5, 6, 7, 8, 9, 10, 11, 12, 13, 14, 15, 16, 17, 18, 19 or 20):

The antibody generated from a mammal can be removed from the serum using the conjugates of the present invention. For example, in one aspect, a compound of Formula II has the structure of Formula IIb:

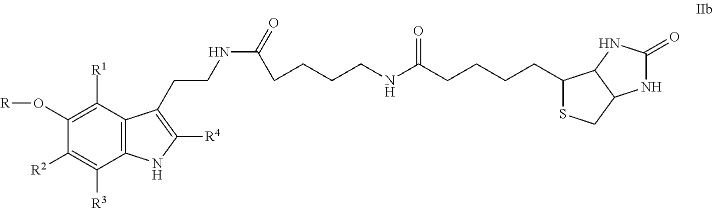

The present invention also provides stable derivatives melatonin and methods for making antibodies. The method comprises:

(a) providing an immunogen comprising a derivative of melatonin (MT);

(b) immunizing an animal with the immunogen under conditions such that the immune system of the animal makes the antibodies; and

(c) removing the antibodies from the animal.

In another aspect, the present invention provides an isolated antibody or antigen binding fragment thereof that specifically binds to melatonin (MT) and has less than 1% cross-reactivity to one or more members selected from the group consisting of tryptophan (Trp), serotonin (5-HT), 5-hydroxytryptophan (5-HTP), 5-hydroxyindole-3-acetic acid (5-HIAA), kynurenine (KYN), kynurenic acid (KYNA), 3-hydroxykynurenine (3-HK), 3-hydroxyanthranilic acid (3-HAA), quinolinic acid (QUIN), anthranilic acid (ANA), serotonin-O-sulfate, and serotonin-O-phosphate.

In certain other aspects, the present invention provides a method for assaying melatonin in a fluid or tissue sample from a mammal, such as a human. The method comprises combining the sample with the antibodies described herein, and then determining whether the antibodies specifically bind to melatonin in the sample. For example, in these methods, specific antibody binding to melatonin from the sample indicates that the melatonin is present in the sample.

In certain instances, the antibodies of the present invention are used in immunoassays such as an Enzyme Linked Immunosorbent Assay (ELISAs, e.g. competitive ELISA) or CEER, which can utilize an enzyme label for the detection of metabolite levels and concentrations.

L represents a linking group for attachment to a biomolecule such as a carrier protein or biotin. In some embodiments, L comprises polyethylene glycol or PEG. For example, L may comprise a terminal amino, carboxylic acid, or sulfhydryl group covalently attached to the ring. In certain instances, the terminal amino, carboxylic acid, or sulfhydryl group is shown and is represented as -L-NH₂, or -L-C(O)OH or -L-SH.

R¹¹ represents the resultant attachment between the compound of the invention and a biomolecule such as a carrier protein, a peptide or biotin (i.e., R¹¹ comprises the linking group joined to a biomolecule).

L is a member selected from the group consisting of a direct link, or a covalent linkage, wherein the covalent linkage is linear or branched, cyclic or heterocyclic, saturated or unsaturated, having 1-60 atoms selected from the group consisting of C, N, P, O, and S, wherein L can have additional hydrogen atoms to fill valences, wherein the linkage contains any combination of ether, thioether, amine, ester, carbamate, urea, thiourea, oxy or amide bonds; or single, double, triple or aromatic carbon-carbon bonds; or phosphorus-oxygen, phosphorus-sulfur, nitrogen-nitrogen, nitrogen-oxygen, or nitrogen-platinum bonds; or aromatic or heteroaromatic bonds. In certain aspects, L comprises a terminal amino, carboxylic acid, or sulfhydryl group.

In certain aspects, L is of the formula:

—X¹—Y¹—X²—

wherein: X¹ is a member selected from the group of a bivalent radical, a direct link, oxygen, an optionally substituted nitrogen and sulfur; Y¹ is a member selected from the group of a direct link and C₁-C₁₀ alkylene optionally interrupted by a heteroatom; and X² is a member selected from the group of a bivalent radical, a direct link, oxygen, an optionally substituted nitrogen and sulfur.

Preferably, the bivalent radical of X¹ and X² are each independently selected from the group of a direct link, optionally substituted alkylene, optionally substituted alkyleneoxycarbonyl, optionally substituted alkylenecarbamoyl, optionally substituted alkylenesulfonyl, arylenesulfonyl, optionally substituted aryleneoxycarbonyl, optionally substituted arylenecarbamoyl, optionally substituted thiocarbonyl, a optionally substituted sulfonyl, and optionally substituted sulfinyl.

In certain preferred aspects, L is —(CH₂)ₙ—, wherein r is an integer from 1 to 10, preferably n is an integer from 1 to 5, such as 1 to 4, or 1, 2, 3, 4, or 5.

In certain instances, a melatonin hapten, a variant thereof, or a derivative thereof and a linker arm L (wherein n is about 1-20) can be used to append a carrier protein to an amino (or sulfhydryl) end. In some embodiments, the linker arm is PEG. The linker arm may include a PEG₁, PEG₂, PEG₃, PEG₄, PEG₅, PEG₆, PEG₇, PEG₈, PEG₉, PEG₁₀, PEG₁₁, PEG₁₂, PEG-₁₃, PEG₁₄, PEG₁₅, PEG₁₆, PEG₁₇, PEG₁₈, PEG₁₉, or PEG20 linker. In one embodiment, a stable melatonin hapten conjugated or linked to a carrier protein, e.g., BSA, RSA, MSA, KLH, OVA and the like to produce an immunogen. In some embodiments, the melatonin hapten is described herein. The hapten can also be conjugated to other biomolecules. For instance, to test the affinity and specificity of the antibody thus produced, a hapten can be linked conjugated or linked to biotin to make a biotinylated hapten e.g., a biotinylated melatonin.

In another aspect, a melatonin compound, a variant thereof, or a derivative thereof can be used to conjugate a carrier protein using conjugation chemistry well known in the art in order to make antibodies. For example, an activated ester (an NHS ester) can react with a primary amine to make a stable amide bond. A maleimide and a thiol can react together and make a thioether. Alkyl halides react with amines and thiols to make alkylamines and thioethers, respectively. Any derivative providing a reactive moiety that can be conjugated to a protein can be utilized herein. As is known in the art, moieties comprising a free amino group, a free carboxylic acid group, or a free sulfhydryl group provide useful reactive groups for protein conjugation. For example, a free amino group can be conjugated to proteins via glutaraldehyde cross-linking, or via carbodiimide cross-linking to available carboxy moieties on the protein. Also, a hapten with a free sulfhydryl group can be conjugated to proteins via maleimide activation of the protein, e.g., using sulfosuccinimidyl-4-(N-maleimidomethyl)cyclohexane-1-carboxylate (Sulfo-SMCC), then linkage to the sulfhydryl group.

When linking a carrier protein having a carboxylic acid group for attachment to an amine containing metabolite, the carboxylic acid can first be converted to a more reactive form using an activating reagent, to form for example, a N-hydroxy succinimide (NHS) ester or a mixed anhydride. The amine-containing metabolite is treated with the resulting activated acid to form an amide linkage. One of skill in the art will recognize that alternatively, the NHS ester can be on the metabolite and the amine can be on the carrier protein.

The present disclosure also provides methods for making antibodies (e.g., antibodies, antibody fragments thereof, and antigen binding fragments thereof) that specifically bind to melatonin, a metabolite of serotonin. The method comprises: (a) providing an immunogen comprising a melatonin hapten conjugated to a carrier protein; (b) immunizing an animal with the immunogen under conditions such that the immune system of the animal makes the antibodies; and (c) removing the antibodies that specifically bind to melatonin from the animal. The animal can be a sheep, goat, rabbit, rat, mouse and the like. In some embodiments, the antibodies are monoclonal antibodies. In other embodiments, the antibodies are polyclonal antibodies. The melatonin hapten can be chemically synthesized or produced by any method known to those skilled in the art.

In one embodiment, the isolated or purified antibodies or antigen binding fragment thereof produced by the method disclosed herein that specifically bind to melatonin have less than 1% cross-reactivity, e.g., 0.9%, 0.8%, 0.7%, 0.6%, 0.5%, 0.4%, 0.3%, 0.2%, 0.1% or 0% cross-reactivity to structurally similar compounds in the tryptophan, serotonin and kynurenine pathways. In some instances, the anti-melatonin antibody or fragment thereof has substantially no cross-reactivity to metabolites or compounds of the tryptophan, serotonin and kynurenine pathways that are structurally similar to melatonin including tryptophan 122, 5-hydroxytryptophan 125, serotonin 101, 5-hydroxyindole acetic acid 115, kynurenine 131, kynurenic acid 135, anthranilic acid 140, 3-hydroxykynurenine 146, 3-hydroxyanthranilic acid 149, quinolinic acid 160, and xanthurenic acid 148 of FIG. 1. Provided herein are polyclonal antibodies and monoclonal antibodies that are specifically immunoreactive to melatonin.

In one aspect, the antibody or antigen binding fragment thereof generated from a mammal can be removed or separated from the serum using the conjugates of the present invention. In some cases, biotinylated melatonin or melatonin conjugated to another biomolecule or compound can be used to remove anti-melatonin antibodies from a mammal. Detailed descriptions of purification methods are disclosed below.

In some aspect, the present invention provides an isolated or purifed monoclonal antibody that is immunoreactive to melatonin and is produced by a hybridoma cell line deposited at American Type Culture Collection (ATCC®) on Nov. 17, 2015 under ATCC Accession No. PTA-122669 and designated 1212-6C1E2F7.

### C. Kynurenine Pathway Metabolite—Kynurenic Acid Haptens

Kynurenine pathway metabolites play a role in the mechanism of visceral pain and have been linked to low level immune activation in IBS. Only 1% of dietary tryptophan is converted to serotonin and more than 95% is metabolized to kynurenines. Both kynurenine levels and the “kynurenine:tryptophan ratio” are significantly increased in patients with IBS. Typically, IBS patients show a decreased concentration of kynurenic acid (KYNA) and an increase in anthranilic acid (ANA) and 3-hydroxyanthranilic acid. Tryptophan metabolism along the kynurenine pathway is inhibited in patient with IBS-D. The present invention provides immunoassays to determine the levels of tryptophan and kynurenine pathway metabolites, which is of diagnostic importance for determining the status of IBS patients.

Provided herein are stable kynurenic acid (KYNA), 3-hydroxykynurenine (3-HK), 3-hydroxyanthranilic acid (3-HAA), quinolinic acid and anthranilic acid haptens that can be conjugated to a biomolecule such as a carrier protein and combined with an adjuvant to stimulate an immune response. The hapten can also be conjugated to other biomolecules. In one embodiment, a stable kynurenic acid (KYNA) hapten conjugated or linked to a carrier protein is produce an immunogen. In some cases, the KYNA hapten is described herein.

The present disclosure also provides methods for making antibodies that specifically bind to a designated kynurenine pathway metabolite such as kynurenic acid (KYNA), a variant thereof, or a derivative thereof. The method comprises: (a) providing an immunogen comprising a hapten selected from the group consisting of kynurenine (K), kynurenic acid (KYNA), 3-hydroxykynurenine (3-HK), 3-hydroxyanthranilic acid (3-HAA), quinolinic acid, anthranilic acid, a variant thereof, or a derivative thereof conjugated to a carrier protein; (b) immunizing an animal with the immunogen under conditions such that the immune system of the animal makes the antibodies; and (c) removing the antibodies from the animal. The animal can be a sheep, goat, rabbit, rat, mouse and the like. In some embodiments, the antibodies are monoclonal antibodies. In other embodiments, the antibodies are polyclonal antibodies.

In one embodiment, the isolated or purified antibodies produced by the method disclosed herein that specifically bind to KYNA have less than 1% cross-reactivity, e.g., 0.9%, 0.8%, 0.7%, 0.6%, 0.5%, 0.4%, 0.3%, 0.2%, 0.1% or 0% cross-reactivity to structurally similar compounds in the tryptophan, serotonin and kynurenine pathways. In some instances, the anti-KYNA antibody has substantially no cross-reactivity to metabolites or compounds of the tryptophan, serotonin and kynurenine pathways that are structurally similar to KYNA including tryptophan 122, 5-hydroxytryptophan 125, serotonin 101, melatonin 120, 5-hydroxyindole acetic acid 115, kynurenine 131, anthranilic acid 140, 3-hydroxykynurenine 146, 3-hydroxyanthranilic acid 149, quinolinic acid 160, and xanthurenic acid 148 of FIG. 1.

In yet another aspect, the present invention provides a compound of Formula III:

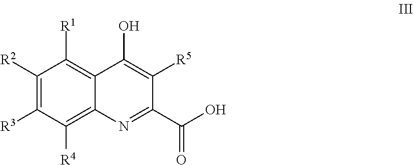

wherein R¹, R², R³, R⁴ and R⁵ are each a member independently selected from the group consisting of hydrogen, alkyl, halo, hydroxyl, alkoxy, amino, aroyl, alkanoyl, amido, substituted amido, cyano, carboxyl, alkoxycarbonyl, sulfonato, alkoxyalkyl, carboxy, carboxyalkyl, alkoxycarbonylalkyl, sulfonatoalkyl L and R¹¹B; L is a linker; R¹¹ is the resultant attachment between the compound and a biomolecule; and B is a biomolecule. The compounds of Formula III are useful in making antibodies specific to kynurenic acid 135.

In another aspect, the compound of Formula III can be used to conjugate a carrier protein using conjugation chemistry well known in the art in order to make antibodies. For example, an activated ester (an NHS ester) can react with a primary amine to make a stable amide bond. A maleimide and a thiol can react together and make a thioether. Alkyl halides react with amines and thiols to make alkylamines and thioethers, respectively. Any derivative providing a reactive moiety that can be conjugated to a protein can be utilized herein. As is known in the art, moieties comprising a free amino group, a free carboxylic acid group, or a free sulfhydryl group provide useful reactive groups for protein conjugation. For example, a free amino group can be conjugated to proteins via glutaraldehyde cross-linking, or via carbodiimide cross-linking to available carboxy moieties on the protein. Also, a hapten with a free sulfhydryl group can be conjugated to proteins via maleimide activation of the protein, e.g., using sulfosuccinimidyl-4-(N-maleimidomethyl)cyclohexane-1-carboxylate (Sulfo-SMCC), then linkage to the sulfhydryl group.

An exemplary schematic for conjugation is as follows, wherein L comprises a

An exemplary embodiment of a kynurenic acid hapten that can be conjugated to a carrier protein. The resulting immunogen can be used to generate a monoclonal or polyclonal antibody against kynurenic acid. In some embodiments, monoclonal antibodies described herein are generated using a kynurenic acid immunogen comprising the chemical structure below. In other embodiments, polyclonal antibodies described herein are generated from a kynurenic acid immunogen comprising the chemical structure below.

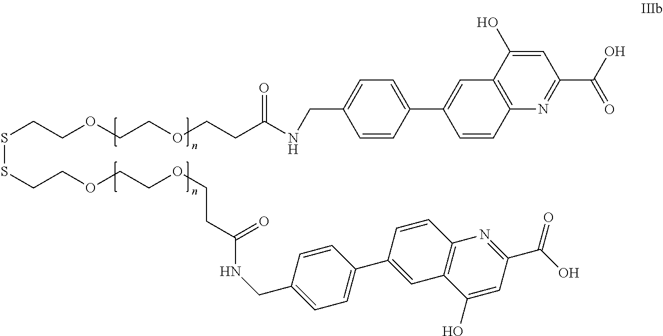

The linker arm (wherein n is about 1-20) can be used to append a carrier protein via thio conjugation.

L represents a linking group for attachment to a biomolecule such as a carrier protein or biotin. In some embodiments, L comprises polyethylene glycol or PEG. For example, L may comprise a terminal amino, carboxylic acid, or sulfhydryl group covalently attached to the ring. In certain instances, the terminal amino, carboxylic acid, or sulfhydryl group is shown and is represented as -L-NH₂, or -L-C(O)OH or -L-SH.

R¹¹ represents the resultant attachment between the compound of the invention and a biomolecule such as a carrier protein, a peptide or biotin (i.e., R¹¹ comprises the linking group joined to a biomolecule).

L is a member selected from the group consisting of a direct link, or a covalent linkage, wherein the covalent linkage is linear or branched, cyclic or heterocyclic, saturated or unsaturated, having 1-60 atoms selected from the group consisting of C, N, P, O, and S, wherein L can have additional hydrogen atoms to fill valences, wherein the linkage contains any combination of ether, thioether, amine, ester, carbamate, urea, thiourea, oxy or amide bonds; or single, double, triple or aromatic carbon-carbon bonds; or phosphorus-oxygen, phosphorus-sulfur, nitrogen-nitrogen, nitrogen-oxygen, or nitrogen-platinum bonds; or aromatic or heteroaromatic bonds. In certain aspects, L comprises a terminal amino, carboxylic acid, or sulfhydryl group.

In certain aspects, L is of the formula:

—X¹—Y¹—X²—

wherein: X¹ is a member selected from the group of a bivalent radical, a direct link, oxygen, an optionally substituted nitrogen and sulfur; Y¹ is a member selected from the group of a direct link and C₁-C₁₀ alkylene optionally interrupted by a heteroatom; and X² is a member selected from the group of a bivalent radical, a direct link, oxygen, an optionally substituted nitrogen and sulfur.

Preferably, the bivalent radical of X¹ and X² are each independently selected from the group of a direct link, optionally substituted alkylene, optionally substituted alkyleneoxycarbonyl, optionally substituted alkylenecarbamoyl, optionally substituted alkylenesulfonyl, arylenesulfonyl, optionally substituted aryleneoxycarbonyl, optionally substituted arylenecarbamoyl, optionally substituted thiocarbonyl, a optionally substituted sulfonyl, and optionally substituted sulfinyl.

In certain preferred aspects, L is —(CH₂)ₙ—, wherein r is an integer from 1 to 10, preferably n is an integer from 1 to 5, such as 1 to 4, or 1, 2, 3, 4, or 5.

The antibody generated from a mammal can be removed from the serum using the conjugates of the present invention. For example, in one aspect, a compound of Formula IIIc, has the structure of Formula III:

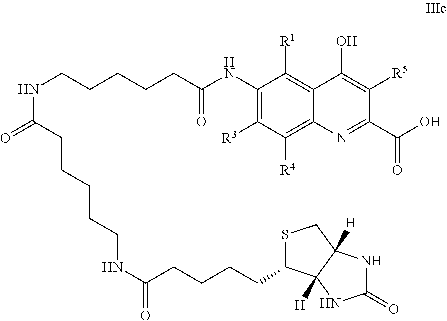

wherein R¹, R³, R⁴, and R⁵ are each hydrogen.

Provided herein is a stable KYNA hapten, a variant thereof, or a derivative thereof that can be conjugated to a biomolecule such as a carrier protein and combined with an adjuvant to stimulate an immune response. In certain instances, a KYNA hapten, a variant thereof, or a derivative thereof and a linker arm (wherein n is about 1-20) can be used to append a carrier protein to an amino (or sulfhydryl) end. In some embodiments, the linker arm is PEG. The linker arm may include a PEG₁, PEG₂, PEG₃, PEG₄, PEG₅, PEG₆, PEG₇, PEG₈, PEG₉, PEG₁₀ , PEG₁₁, PEG₁₂, PEG₁₃, PEG₁₄, PEG₁₅, PEG₁₆, PEG₁₇ , PEG₁₈, PEG₁₉, or PEG₂₀ linker. In one embodiment, a stable KYNA hapten conjugated or linked to a carrier protein, e.g., BSA, RSA, MSA, KLH, OVA and the like to produce an immunogen. The hapten can also be conjugated to other biomolecules. For instance, to test the affinity and specificity of the antibody thus produced, a hapten can be linked conjugated or linked to biotin to make a biotinylated hapten e.g., a biotinylated KYNA.

In another aspect, the present invention provides an isolated or purified antibody or antigen binding fragment thereof that specifically binds to kynurenic acid, and wherein the antibody has less than 1% cross-reactivity to one or more members selected from the group consisting of tryptophan 122, 5-hydroxytryptophan 125, serotonin 101, 5-hydroxyindole acetic acid (5-HIAA) 115, kynurenine 131, anthranilic acid 140, 3-hydroxykynurenine 146, 3-hydroxyanthranilic acid 149, quinolinic acid 160, xanthurenic acid 148 and melatonin 120 of FIG. 1

In some aspect, the present invention provides an isolated or purifed monoclonal antibody that is immunoreactive to KYNA and is produced by a hybridoma cell line deposited at American Type Culture Collection (ATCC®) on Nov. 17, 2015 under ATCC Accession No. PTA-122670 and designated 1194-6H5B11A7. Such an antibody has substantially no cross-reactivity to other structurally similar metabolites or compounds of the tryptophan, serotonin, and kynurenine pathway.

### D. Detecting Metabolites in a Biological Sample Using an Immunoassay

In some aspects, the present disclosure provides assay methods and kits for detecting, measuring or quantitating the level of melatonin in a biological sample from a subject, such as a human subject. In some embodiments, the human subject has a condition associated with a higher or lower level of melatonin, 5-HIAA, and/or kynurenic acid compared to a normal subject. In some instances, the condition is irritable bowel syndrome including any one of the subtypes: IBS with constipation (IBS-C), IBS with diarrhea (IBS-D), mixed IBS (IBS-M) and unsubtyped IBS (IBS-U). The method can include using an antibody against 5-HIAA, biotinylated 5-HIAA, an antibody against kynurenic acid, biotinylated kynurenic acid, an antibody against melatonin, biotinylated melatonin, and any combination thereof.

In some aspects, the present invention provides a method for assaying, measuring or detecting the presence or level of a serotonin metabolite in a biological sample such as a fluid or tissue sample from a mammal, e.g., a human. In some embodiments, the serotonin metabolite is 5-hydroxyindole acetic acid (5-HIAA). In some instances, the method includes measuring or quantitating the amount or concentration of 5-HIAA in a biological sample obtained from a human subject. The method can comprise combining the sample with an antibody that specifically binds to 5-HIAA under conditions to form a complex between the antibody and 5-HIAA if present in the sample. The antibody can be any of the anti-5HIAA antibodies described herein. In some embodiments, the sample and the anti-5HIAA antibody are also combined with an immobilized 5-HIAA derivative. The immobilized 5-HIAA derivative may be biotinylated 5-HIAA as described herein that has been attached or bound to a streptavidin-coated solid substrate such as a streptavidin-coated multiwell plate. In some embodiments, the sample, the anti-5HIAA antibody, and immobilized 5-HIAA derivative are simultaneously contacted or added together. In some cases, the sample and the anti-5-HIAA antibody are incubated together for a preselected duration, and then incubated with immobilized 5-HIAA or biotinylated 5-HIAA. In other cases, the immobilized or biotinylated 5-HIAA derivative is incubated with the anti-5-HIAA antibody for a preselected duration, and then incubated with the sample. In yet other cases, the sample, the anti-5HIAA antibody and immobilized 5HIAA are contacted together sequentially in any order. The level of the 5-HIAA in the sample can be determined by measuring the level of anti-5-HIAA antibody bound to the immobilized 5-HIAA derivative, and calculating the corresponding level of 5-HIAA in the sample. In other words, the level of anti-5HIAA antibody complexed with the immobilized 5-HIAA derivative can be measured directly and the level of 5-HIAA in the sample is quantitated indirectly. In some cases, there is an inverse proportion of 5-HIAA in the sample compared to the level of anti-5-HIAA antibody bound to the immobilized 5-HIAA derivative.

In other aspects, the present invention provides a method for assaying the presence or level of a serotonin metabolite such as melatonin in a biological sample such as a fluid or tissue sample from a mammal, e.g., a human subject. In some embodiments, the method comprise combining the sample obtained from the subject with an antibody that specifically binds to melatonin under conditions to form a complex between the antibody and melatonin if present in the sample. The antibody can be any anti-melatonin antibody disclosed herein. In some embodiments, the sample and the anti-melatonin antibody are also combined with immobilized melatonin. The immobilized melatonin may be biotinylated melatonin as described herein that has been attached or bound to a streptavidin-coated solid substrate such as a streptavidin-coated multiwell plate. In some embodiments, the sample, the anti-antibody, and immobilized melatonin are simultaneously contacted or added together. In some cases, the sample and the anti-melatonin antibody are incubated together for a preselected duration, and then incubated with immobilized melatonin or biotinylated melatonin. In other cases, the immobilized melatonin or biotinylated melatonin are incubated with the anti-melatonin antibody for a preselected duration, and then incubated with the sample. In yet other cases, the sample, the anti-melatonin antibody and immobilized melatonin are contacted together sequentially in any order. The level of the melatonin in the sample can be determined by measuring the level of anti-melatonin antibody bound to the immobilized melatonin, and calculating the corresponding level of melatonin in the sample. For instance, the level of anti-melatonin antibody complexed with the immobilized melatonin can be measured directly and the level of melatonin in the sample can be quantitated indirectly. In some cases, there is an inverse proportion of melatonin in the sample compared to the level of anti-melatonin antibody bound to immobilized melatonin.

In other aspects, the present invention provides a method for assaying the presence or level of a kynurenine metabolite such as kynurenic acid (KYNA) in a biological sample such as a fluid or tissue sample from a mammal, e.g., a human subject. In some embodiments, the method comprise combining the sample obtained from the subject with an antibody that specifically binds to KYNA under conditions to form a complex between the antibody and melatonin if present in the sample. The antibody can be any anti-KYNA antibody disclosed herein. In some embodiments, the sample and the anti-KYNA antibody are also combined with immobilized KYNA. The immobilized KYNA may be biotinylated KYNA as described herein that has been attached or bound to a streptavidin-coated solid substrate such as a streptavidin-coated multiwell plate. In some embodiments, the sample, the anti-antibody, and immobilized KYNA are simultaneously contacted or added together. In some cases, the sample and the anti-KYNA antibody are incubated together for a preselected duration, and then incubated with immobilized KYNA or biotinylated KYNA. In other cases, the immobilized or biotinylated KYNA are incubated with the anti-KYNA antibody for a preselected duration, and then incubated with the sample. In yet other cases, the sample, the anti-KYNA antibody and immobilized KYNA are contacted together sequentially in any order. The level of the KYNA in the sample can be determined by measuring the level of anti-KYNA antibody bound to the immobilized KYNA, and calculating the corresponding level of KYNA in the sample. In some embodiments, the level of anti-KYNA antibody complexed with immobilized KYNA can be measured directly and the level of KYNA in the sample can be quantitated indirectly. In some cases, there is an inverse proportion of KYNA in the sample compared to the level of anti-KYNA antibody bound to immobilized KYNA.

In some embodiments, the sample is a whole blood sample, a plasma sample, or a serum sample. Such samples can be isolated or obtained from a subject, such as a human subject. In some cases, the subject has been diagnosed as having IBS. In other cases, the subject has not been diagnosed with IBS. In some instances, the subject is suspected of having IBS. In other instances, the subject is experiencing or exhibiting one or more symptoms of IBS. In some embodiments, the sample used in the assay method is a diluted sample. The sample may be an unprocessed sample. In some instances, the volume of the sample used in the method is less than about 100 μL, e.g., about 99 μL, 90 μL, 85 μL, 80 μL, 75 μL, 70 μL, 65 μL, 60 μL, 55 μL, 50 μL, 45 μL, 40 μL, 35 μL, 30 μL, 25 μL, 20 μL, 15 μL, 10 μL, 5 μL, or less. The sample volume can be less than about 50 μL, e.g., about 50 μL, 45 μL, 40 μL, 35 μL, 30 μL, 25 μL, 20 μL, 15 μL, 10 μL, 5 μL, or less.

In some embodiments, the assay method takes less than 24 hours to perform, e.g., 23 hrs, 22 hrs, 21 hrs, 20 hrs, 19 hrs, 18 hrs, 17 hrs, 16 hrs, 15 hrs, 14 hrs, 13 hrs, 12 hrs, 11 hrs, 10 hrs, 9 hrs, 8 hrs, 7 hrs, 6 hrs, 5 hrs, 4 hrs, 3 hrs, 2 hrs, 1 hr, 30 minutes or less to perform.

In certain aspects, the step of measuring the level of bound anti-metabolite antibody or the level of a metabolite is performed using an immunoassay. Immunoassays provide reliable and facile ways to monitor metabolites in biological fluids. The present invention provides reliable immunoassays of high specificity and sensitivity for the detection and quantification of one or more tryptophan, serotonin, kynurenine metabolites. In some embodiments, the immunoassay are an enzyme linked immunosorbent assay (ELISA), e.g., a competitive ELISA or a proximity immunoassay, e.g., CEER™.

In some embodiments, the antibodies described herein can be conjugated to any detectable label or moiety that can be used to measure the formed antigen-antibody complex.

In some cases, the antibody is directly conjugated to a readable signal such as chromophores, colloidal gold, colored latex, fluorophores and the like. In other cases, the antibody is conjugated to an enzyme, peptide or other biomolecule.

In one aspect, the present invention provides assay methods wherein an antibody-antigen reaction is carried out. In one embodiment of an ELISA, an antigen or metabolite such as 5-HIAA, melatonin or KYNA present in a sample obtained from a subject is allowed to react with an enzyme-labeled, e.g., peroxidase-labeled antibody specific to the metabolite being assayed to form an antigen-antibody complex. The thus formed antigen-antibody complex is then allowed to react with a detection substrate, so that the activity of the enzyme, e.g., peroxidase or phosphatase is measured. In some embodiments, the antibody specific to the metabolite is not enzyme labeled and an enzyme-labeled secondary antibody that recognized the antibody specific to the metabolite is used. A detection substrate can be used to react with the enzyme label of the secondary antibody in order to measure the activity of the enzyme. The enzyme-labeled antibody can be an alkaline phosphatase-, β-galactosidase-, or HRP-labeled antibody.

Any detection substrate recognized by those skilled in the art can be used. For instance, for a chemiluminescent reaction, the substrate can be luminol, Supersignal® ELISA Pico chemiluminescent substrate (Thermo Fisher), and DynaLight™ chemiluminescent substrate (Thermo Fisher). For a colorimetric reaction, a substrate such as 4-chloro-1-napthol, p-nitrophenyl phosphate (PNPP), OPD, ONPG, or TMB can be used. A substrate such as 4-methylumbelliferyl phosphate disodium salt (MUP), QuantaBlu™ Fluorogenic substrate (Thermo Fisher), and Amplex® Red Reagent (Thermo Fisher) can be used for a fluorescent reaction. The presence, concentration and or level of the metabolite can thereby be measured using, for example, a spectrometer or other detection device.

In another ELISA embodiment, the metabolite or a derivative thereof can be immobilized. An antibody of the present invention can be used to bind to the immobilized metabolite to form an antigen-antibody complex. A sample that contains the metabolite can be used to compete for antibody-antigen binding. Thereafter, the conjugate can be detected by another antibody (secondary antibody) with an enzyme label. The enzyme label is then reacted with detection reagents or substrates, and then monitored. In other cases, the antibody of the present invention is conjugated to a detectable moiety or label and can be reacted and/or detected without using a secondary antibody.

The assay methods to detect any of the metabolites described herein can comprise any immunoassay known in the art. In some aspects, the assay is performed in a liquid phase. In other embodiments, the assay is performed on a solid phase or solid support, e.g., on a bead or a microplate, for example a 96 well microtiter plate. Non-limiting examples of immunoassays useful in these methods are a radioimmunoassay, a microarray assay, a fluorescence polarization immunoassay, an immunoassay comprising FRET, enzyme linked immunosorbent assay (ELISA) or CEER™.

Any ELISA known in the art as useful for hapten detection can be utilized for the instant assays. ELISA for haptens generally utilize a competitive format, i.e., where the hapten (a metabolite) in the sample competes with a labeled hapten (e.g., a biotin-hapten or enzyme-hapten conjugate) for anti-hapten antibody binding sites such that less labeled hapten is bound when there is more hapten in the sample. Thus, in these competitive assays, an increasing amount of hapten in the sample results in less enzyme bound to the solid phase, and consequently less detectable signal. In such competitive assays the sample can be added with the labeled hapten to compete directly for antibody binding sites, or the sample and labeled hapten can be added sequentially such that the labeled hapten simply binds where the sample hapten is not bound. In some embodiments, the ELISA is a direct competitive ELISA, or an indirect competitive ELISA.

In one embodiment, the antibodies produced herein are bound to a solid phase, either directly or indirectly, the latter being where the solid phase is coated with an anti-antibody (for example goat antibodies that bind to rabbit IgG antibodies (goat anti-rabbit IgG) and the antibodies are bound to the anti-antibody. The anti-antibodies are also known as secondary antibodies. In these assays, the sample and a labeled hapten are added to the solid phase to compete with antibody binding sites on the coated solid phase. After washing, the signal is generated, which measures the amount of labeled hapten that is bound to the solid phase.

Provided herein are kits for performing the assay methods described above. In some embodiments, the kit comprises an antibody that specifically binds to 5-HIAA, e.g., an anti-5HIAA monoclonal antibody or polyclonal antibody, and optionally a biotinylated 5-HIAA derivative. The monoclonal antibody against 5-HIAA may be produced by the hybridoma clone having the ATCC Accession No. PTA-122671, deposited on Nov. 17, 2015, and designated 1204-10G6F11H3.

In other embodiments, the kit comprises an antibody that specifically binds to melatonin, e.g., an anti-melatonin monoclonal antibody or polyclonal antibody, and optionally biotinylated melatonin. The monoclonal antibody may be produced by the hybridoma clone having the ATCC Accession No. PTA-122669, deposited on Nov. 17, 2015, and designated 1212-6C1E2F7.

In yet other embodiments, the kit comprises an antibody that specifically binds to kynurenic acid, e.g., an anti-kynurenic acid monoclonal antibody or polyclonal antibody, and optionally biotinylated kynurenic acid. The monoclonal antibody may be produced by the hybridoma clone having the ATCC Accession No. PTA-122670, deposited on Nov. 17, 2015, and designated 1194-6H5B11A7.

In some instances, the kit also includes an instruction manual for performing the assay methods discussed herein. The kit may include a standard control metabolite such as a 5-HIAA standard control, melatonin standard control or a kynurenic acid standard control. In some embodiments, the standard control metabolite comprises a preselected or known concentration of the metabolite of interest.

### E. Polyclonal Antibodies

Polyclonal antibodies provided herein can be of any isotype such as one of the major antibody isotypes: IgA, IgD, IgE, IgG, and IgM. In some embodiments, the antibody can be classified as an IgG₁, IgG₂, IgG₃, IgG₄, IgA₁ or IgA₂ antibody. In some instances, the antibody has a kappa (κ) light chain or a lambda (λ) light chain.

Polyclonal antibodies are preferably raised in animals by multiple subcutaneous (sc) or intraperitoneal (ip) injections of an antigen of the present invention and an adjuvant. It may be useful to conjugate the antigen of interest to a carrier protein that is immunogenic in the species to be immunized using a bifunctional or derivatizing agent. Non-limiting examples of bifunctional or derivatizing agents include maleimidobenzoyl sulfosuccinimide ester (conjugation through cysteine residues), N-hydroxysuccinimide (conjugation through lysine residues), glutaraldehyde, succinic anhydride, SOCl₂, and R¹N═C═NR, wherein R and R¹ are different alkyl groups.

Animals are immunized against the antigens of the present invention or an immunogenic conjugate or derivative thereof by combining, e.g., 100 μg (for rabbits) or 5 μg (for mice) of the antigen or conjugate with 3 volumes of Freund's complete adjuvant and injecting the solution intradermally at multiple sites. One month later, the animals are boosted with about ⅕ to 1/10 the original amount of conjugate in Freund's incomplete adjuvant by subcutaneous injection at multiple sites. Seven to fourteen days later, the animals are bled and the serum is assayed for antibody titer. Animals are typically boosted until the titer plateaus. Preferably, the animal is boosted with the conjugate of the same antigen, but conjugation to a different immunogenic antigen and/or through a different cross-linking reagent may be used. In certain instances, aggregating agents such as alum can be used to enhance the immune response. Detailed descriptions of methods for producing polyclonal antibodies is found in, e.g., Antibodies, A Laboratory Manual, Harlow and Lane, Eds., Cold Spring Harbor Laboratory, Cold Spring Harbor, N.Y. (1988).

### F. Monoclonal Antibodies

Monoclonal antibodies provided herein can be of any isotype such as one of the major antibody isotypes: IgA, IgD, IgE, IgG, and IgM. In some embodiments, the antibody can be classified as an IgG₁, IgG₂, IgG₃, IgG₄, IgA₁ or IgA₂ antibody. In some instances, the antibody has a kappa (κ) light chain or a lambda (λ) light chain. In some embodiments, the monoclonal antibody against 5-HIAA or the monoclonal antibody produced by hybridoma clone having ATCC Accession No. PTA-122671, deposited on Nov. 17, 2015 and designated 1204-10G6F11H3 is an IgG1κ antibody. In other embodiments, the monoclonal antibody against melatonin or the monoclonal antibody produced by hybridoma clone having ATCC Accession No. PTA-122669, deposited on Nov. 17, 2015 and designated 1212-6C1E2F7 is an IgG₃κ antibody. In yet other embodiments, the monoclonal antibody against KYNA or the monoclonal antibody produced by hybridoma clone having ATCC Accession No. PTA-122670, deposited on Nov. 12, 2015 and designated 1194-6H5B11A7 is an IgG1κ antibody.

Monoclonal antibodies are generally obtained from a population of substantially homogeneous antibodies, i.e., the individual antibodies comprising the population are identical except for possible naturally-occurring mutations that may be present in minor amounts. Thus, the modifier “monoclonal” indicates the character of the antibody as not being a mixture of discrete antibodies. For example, monoclonal antibodies can be made using the hybridoma method described by Kohler et al., Nature, 256:495 (1975) or by any recombinant DNA method known in the art (see, e.g., U.S. Pat. No. 4,816,567).

In the hybridoma method, a mouse or other appropriate host animal (e.g., hamster) is immunized as described above to elicit lymphocytes that produce or are capable of producing antibodies which specifically bind to the polypeptide of interest used for immunization. Alternatively, lymphocytes are immunized in vitro. The immunized lymphocytes are then fused with myeloma cells using a suitable fusing agent, such as polyethylene glycol, to form hybridoma cells (see, e.g., Goding, Monoclonal Antibodies: Principles and Practice, Academic Press, pp. 59-103 (1986)). The hybridoma cells thus prepared are seeded and grown in a suitable culture medium that preferably contains one or more substances which inhibit the growth or survival of the unfused, parental myeloma cells. For example, if the parental myeloma cells lack the enzyme hypoxanthine guanine phosphoribosyl transferase (HGPRT), the culture medium for the hybridoma cells will typically include hypoxanthine, aminopterin, and thymidine (HAT medium), which prevent the growth of HGPRT-deficient cells.

Preferred myeloma cells are those that fuse efficiently, support stable high-level production of antibody by the selected antibody-producing cells, and/or are sensitive to a medium such as HAT medium. Examples of such preferred myeloma cell lines for the production of human monoclonal antibodies include, but are not limited to, murine myeloma lines such as those derived from MOPC-21 and MPC-11 mouse tumors (available from the Salk Institute Cell Distribution Center; San Diego, Calif.), SP-2 or X63-Ag8-653 cells (available from the American Type Culture Collection; Rockville, Md.), and human myeloma or mouse-human heteromyeloma cell lines (see, e.g., Kozbor, J. Immunol., 133:3001 (1984); and Brodeur et al., Monoclonal Antibody Production Techniques and Applications, Marcel Dekker, Inc., New York, pp. 51-63 (1987)).

The culture medium in which hybridoma cells are growing can be assayed for the production of monoclonal antibodies directed against the polypeptide of interest. Preferably, the binding specificity of monoclonal antibodies produced by hybridoma cells is determined by immunoprecipitation or by an in vitro binding assay, such as a radioimmunoassay (RIA) or an enzyme-linked immunoabsorbent assay (ELISA). The binding affinity of monoclonal antibodies can be determined using, e.g., the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (see, e.g., Goding, Monoclonal Antibodies: Principles and Practice, Academic Press, pp. 59-103 (1986)). Suitable culture media for this purpose include, for example, D-MEM or RPMI-1640 medium. In addition, the hybridoma cells may be grown in vivo as ascites tumors in an animal. The monoclonal antibodies secreted by the subclones can be separated from the culture medium, ascites fluid, or serum by conventional antibody purification procedures such as, for example, protein A-Sepharose, hydroxylapatite chromatography, gel electrophoresis, dialysis, or affinity chromatography.

DNA encoding the monoclonal antibodies can be readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody, to induce the synthesis of monoclonal antibodies in the recombinant host cells. See, e.g., Skerra et al., Curr. Opin. Immunol., 5:256-262 (1993); and Pluckthun, Immunol Rev., 130:151-188 (1992). The DNA can also be modified, for example, by substituting the coding sequence for human heavy chain and light chain constant domains in place of the homologous murine sequences (see, e.g., U.S. Pat. No. 4,816,567; and Morrison et al., Proc. Natl. Acad. Sci. USA, 81:6851 (1984)), or by covalently joining to the immunoglobulin coding sequence all or part of the coding sequence for a non-immunoglobulin polypeptide.

In a further embodiment, monoclonal antibodies or antibody fragments thereof can be isolated from antibody phage libraries generated using the techniques described in, for example, McCafferty et al., Nature, 348:552-554 (1990); Clackson et al., Nature, 352:624-628 (1991); and Marks et al., J. Mol. Biol., 222:581-597 (1991). The production of high affinity (nM range) human monoclonal antibodies by chain shuffling is described in Marks et al., BioTechnology, 10:779-783 (1992). The use of combinatorial infection and in vivo recombination as a strategy for constructing very large phage libraries is described in Waterhouse et al., Nuc. Acids Res., 21:2265-2266 (1993). Thus, these techniques are viable alternatives to traditional monoclonal antibody hybridoma methods for the generation of monoclonal antibodies.

### G. Antibody Fragments

Various techniques have been developed for the production of antibody fragments. Traditionally, these fragments were derived via proteolytic digestion of intact antibodies (see, e.g., Morimoto et al., J. Biochem. Biophys. Meth., 24:107-117 (1992); and Brennan et al., Science, 229:81 (1985)). However, these fragments can now be produced directly using recombinant host cells. For example, the antibody fragments can be isolated from the antibody phage libraries discussed above. Alternatively, Fab′-SH fragments can be directly recovered from E. coli cells and chemically coupled to form F(ab′)₂ fragments (see, e.g., Carter et al., BioTechnology, 10:163-167 (1992)). According to another approach, F(ab′)₂ fragments can be isolated directly from recombinant host cell culture. Other techniques for the production of antibody fragments will be apparent to those skilled in the art. In other embodiments, the antibody of choice is a single chain Fv fragment (scFv). See, e.g., PCT Publication No. WO 93/16185; and U.S. Pat. Nos. 5,571,894 and 5,587,458. The antibody fragment may also be a linear antibody as described, e.g., in U.S. Pat. No. 5,641,870. Such linear antibody fragments may be monospecific or bispecific.

### H. Bispecific Antibodies

Bispecific antibodies are antibodies that have binding specificities for at least two different epitopes. Exemplary bispecific antibodies may bind to two different epitopes of the same polypeptide of interest. Other bispecific antibodies may combine a binding site for the polypeptide of interest with binding site(s) for one or more additional antigens. Bispecific antibodies can be prepared as full-length antibodies or antibody fragments (e.g., F(ab′)₂ bispecific antibodies).

Methods for making bispecific antibodies are known in the art. Traditional production of full-length bispecific antibodies is based on the co-expression of two immunoglobulin heavy chain-light chain pairs, where the two chains have different specificities (see, e.g., Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule is usually performed by affinity chromatography. Similar procedures are disclosed in PCT Publication No. WO 93/08829 and Traunecker et al., EMBO 1, 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy chain constant region (CH1) containing the site necessary for light chain binding present in at least one of the fusions. DNA encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains into one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. This asymmetric structure facilitates the separation of the desired bispecific compound from unwanted immunoglobulin chain combinations, as the presence of an immunoglobulin light chain in only one half of the bispecific molecule provides for a facile way of separation. See, e.g., PCT Publication No. WO 94/04690 and Suresh et al., Meth. Enzymol., 121:210 (1986).

According to another approach described in U.S. Pat. No. 5,731,168, the interface between a pair of antibody molecules can be engineered to maximize the percentage of heterodimers which are recovered from recombinant cell culture. The preferred interface comprises at least a part of the CH3 domain of an antibody constant domain. In this method, one or more small amino acid side-chains from the interface of the first antibody molecule are replaced with larger side chains (e.g., tyrosine or tryptophan). Compensatory “cavities” of identical or similar size to the large side-chain(s) are created on the interface of the second antibody molecule by replacing large amino acid side-chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Heteroconjugate antibodies can be made using any convenient cross-linking method. Suitable cross-linking agents and techniques are well-known in the art, and are disclosed in, e.g., U.S. Pat. No. 4,676,980.

Suitable techniques for generating bispecific antibodies from antibody fragments are also known in the art. For example, bispecific antibodies can be prepared using chemical linkage. In certain instances, bispecific antibodies can be generated by a procedure in which intact antibodies are proteolytically cleaved to generate F(ab′)₂ fragments (see, e.g., Brennan et al., Science, 229:81 (1985)). These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody.

In some embodiments, Fab′-SH fragments can be directly recovered from E. coli and chemically coupled to form bispecific antibodies. For example, a fully humanized bispecific antibody F(ab′)₂ molecule can be produced by the methods described in Shalaby et al., J. Exp. Med., 175: 217-225 (1992). Each Fab′ fragment was separately secreted from E. coli and subjected to directed chemical coupling in vitro to form the bispecific antibody.

Various techniques for making and isolating bispecific antibody fragments directly from recombinant cell culture have also been described. For example, bispecific antibodies have been produced using leucine zippers. See, e.g., Kostelny et al., J. Immunol., 148:1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy chain variable domain (VH) connected to a light chain variable domain (VL) by a linker which is too short to allow pairing between the two domains on the same chain. Accordingly, the VH and VL domains of one fragment are forced to pair with the complementary VL and VH domains of another fragment, thereby forming two antigen binding sites. Another strategy for making bispecific antibody fragments by the use of single-chain Fv (sFv) dimers is described in Gruber et al., J. Immunol., 152:5368 (1994).

### I. Antibody Purification

The antibodies can be purified by methods known to the skilled artisan. Purification methods include, among others, selective precipitation, liquid chromatography, HPLC, electrophoresis, chromatofocusing, gel electrophoresis, dialysis, and various affinity techniques. Selective precipitation may use ammonium sulfate, ethanol (Cohn precipitation), polyethylene glycol, or others available in the art. Liquid chromatography mediums, include, among others, ion exchange medium DEAE, polyaspartate), hydroxylapatite, size exclusion (e.g., those based on crosslinked agarose, acrylamide, dextran, etc.), hydrophobic matrixes (e.g., Blue Sepharose). Affinity techniques typically rely on proteins that interact with the immunoglobulin Fc domain. Protein A from Staphylococcus aureas can be used to purify antibodies that are based on human γ1, γ2, or γ4 heavy chains (Lindmark et al., J. Immunol. Meth. 62:1-13 (1983)). Protein G from C and G streptococci is useful for all mouse isotypes and for human γ3 (Guss et al., EMBO J. 5:15671575 (1986)). Protein L, a Peptostreptococcus magnus cell-wall protein that binds immunoglobulins (Ig) through k light-chain interactions (BD Bioscience/ClonTech. Palo Alto, Calif.), is useful for affinity purification of Ig subclasses IgM, IgA, IgD, IgG, IgE and IgY. Recombinant forms of these proteins are also commercially available. If the antibody contains metal binding residues, such as phage display antibodies constructed to contain histidine tags, metal affinity chromatography may be used.

When sufficient amounts of specific cell populations are available, antigen affinity matrices may be made with the cells to provide an affinity method for purifying the antibodies. The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker; Phillipsburg, N.J.) can be useful for purification. Other techniques for protein purification such as fractionation on an ion-exchange column, ethanol precipitation, reverse phase HPLC, chromatography on silica, chromatography on heparin SEPHAROSE™, chromatography on an anion or cation exchange resin (such as a polyaspartic acid column), chromatofocusing, SDS-PAGE, and ammonium sulfate precipitation are also available depending on the antibody to be recovered.

When using recombinant techniques, antibodies can be produced inside an isolated host cell, in the periplasmic space of a host cell, or directly secreted from a host cell into the medium. If the antibody is produced intracellularly, the particulate debris is first removed, for example, by centrifugation or ultrafiltration. Carter et al., BioTech., 10:163-167 (1992) describes a procedure for isolating antibodies which are secreted into the periplasmic space of E. coli. Briefly, cell paste is thawed in the presence of sodium acetate (pH 3.5), EDTA, and phenylmethylsulfonylfluoride (PMSF) for about 30 min. Cell debris can be removed by centrifugation. Where the antibody is secreted into the medium, supernatants from such expression systems are generally concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25 M salt).

One of skill in the art will appreciate that any binding molecule having a function similar to an antibody, e.g., a binding molecule or binding partner which is specific for one or more analytes of interest in a sample, can also be used in the methods and compositions of the present invention. Examples of suitable antibody-like molecules include, but are not limited to, domain antibodies, unibodies, nanobodies, shark antigen reactive proteins, avimers, adnectins, anticalms, affinity ligands, phylomers, aptamers, affibodies, trinectins, and the like.

### J. Methods for Assessing Reactivity of Isolated Antibodies

The generation and selection of antibodies can be accomplished several ways. The synthesized and purified antigen corresponding to the metabolite of interest is injected, for example, into mice or rabbits or another mammal, to generate polyclonal or monoclonal antibodies. One skilled in the art will recognize that many procedures are available for the production of antibodies, for example, as described in Antibodies, A Laboratory Manual, Harlow and Lane, Eds., Cold Spring Harbor Laboratory, Cold Spring Harbor, N.Y. (1988). One skilled in the art will also appreciate that binding fragments or Fab fragments which mimic (e.g., retain the functional binding regions of) antibodies can also be prepared from genetic information by various procedures. See, e.g., Antibody Engineering: A Practical Approach, Borrebaeck, Ed., Oxford University Press, Oxford (1995); and Huse et al., J. Immunol., 149:3914-3920 (1992).

The antibodies that are generated by these methods can then be selected by first screening for affinity and specificity with the purified antigen of interest (such as the biotinylated haptens described herein) and, if required, comparing the results to the affinity and specificity of the antibodies with other antigens that are desired to be excluded from binding. The screening procedure can involve immobilization of the purified antigens in separate wells of microtiter plates. The plates can have streptavidin immobilized thereon and the solution containing a potential antibody or group of antibodies is then placed into the respective microtiter wells and incubated for about 30 minutes to 2 hours. The microtiter wells are then washed and a labeled secondary antibody (e.g., an anti-mouse antibody conjugated to alkaline phosphatase if the raised antibodies are mouse antibodies) is added to the wells and incubated for about 30 minutes and then washed. Substrate is added to the wells and a color reaction will appear where antibody to the immobilized antigen, such as the biotinylated antigen, is present.

The antibodies so identified can then be further analyzed for affinity and specificity. In the development of immunoassays for a target metabolite, the purified target metabolite acts as a standard with which to judge the sensitivity and specificity of the immunoassay using the antibodies that have been selected. Because the binding affinity of various antibodies may differ, e.g., certain antibody combinations may interfere with one another sterically, assay performance of an antibody can be a more important measure than absolute affinity and specificity of that antibody.

Those skilled in the art will recognize that many approaches can be taken in producing antibodies or binding fragments and screening and selecting for affinity and specificity for the various metabolites of interest, but these approaches do not change the scope of the present invention.

### III. Methods of Use

The present invention provides a method for determining a diagnosis of irritable bowel syndrome (IBS) in a subject using the presence or concentrations (amounts or levels) of the metabolites herein. The method may comprise measuring one or more metabolites in blood, plasma or serum obtained from a patient by the assay methods described herein.

The present invention also provides a method for determining whether a patient is responding to a treatment for, e.g., IBS. The method may comprise measuring one or more metabolites in blood, plasma or serum of the patient by the assay methods described herein. In some embodiments, the efficacy of a treatment is predicted based on the level of 5-HIAA, melatonin and/or kynurenic acid in a biological sample from an IBS patient before or after administration of the treatment. The method is useful for determining whether an IBS patient has had a clinical response to the treatment.

In certain other aspects, the present invention provides a method for evaluating a patient previously diagnosed with IBS or prognosing an IBS patient. The method comprises measuring one or more metabolites in blood, plasma or serum of the patient by an assay method described herein. In some embodiments, the method includes measuring the level of 5-HIAA, melatonin and/or kynurenic acid in a biological sample from an IBS patient at one time point, measuring the level of 5-HIAA, melatonin and/or kynurenic acid in a second biological sample from the patient at a second time point, and calculating the change or difference between the levels at the two time points. The method can also include using a statistical algorithm to predict the likelihood that the patient has less or more severe IBS compared to before (e.g., the initial diagnosis of IBS). In some cases, a statistical algorithm can be used to predict the patient's IBS subtype.

The following examples are offered to illustrate, but not to limit the claimed invention.

### Indirect Competitive ELISA Assays for Pathway Metabolites

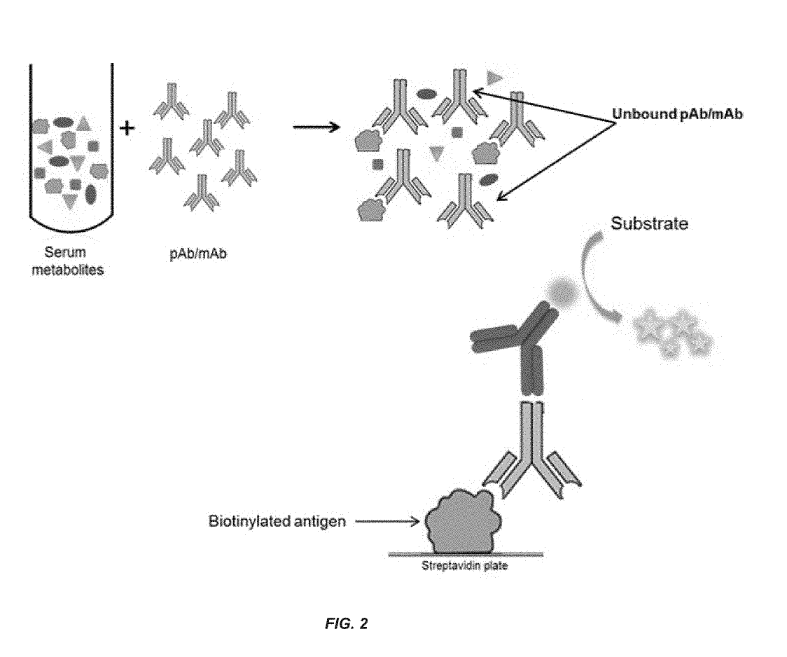

This example describes the use of isolated antibodies that specifically bind to metabolites provided herein, such as metabolites in the tryptophan, serotonin, and kynurenine pathways (FIG. 1). This example also shows that these antibodies can be used in competitive ELISA assays to accurately and effectively detect, measure and quantitate specific metabolites in samples, e.g., patient serum (FIG. 2). The antibodies exhibited no cross-reactivity (or substantially no cross-reactivity) detection. The competitive ELISA provides an accurate, quantitative measure of metabolite concentrations.

The competitive ELISA is based on novel antibodies raised to the synthetically made metabolite analogs (haptens) which serve as the immunogenic conjugate (e.g., antigens). The analogs were specifically designed with a linker to project the small molecule and elicit an immune response specific to the hapten.

### Biotinylated Hapten

A biotinylated hapten was generated for each pathway metabolite or derivative thereof. Instead of conjugating the linker arm to a carrier protein, the linker was conjugated to biotin. For example, a biotinylated benzoxazole derivative of 5-HIAA was chemically synthesized to contain a linker arm at the phenyl end of the derivative and biotin at the other end of the linker.

### Competitive ELISA

FIG. 2 provides an exemplary embodiment of the competitive ELISA that was be used to detect a pathway metabolite in a patient's serum. The assay plate was made by coating a streptavidin plate with the biotinylated hapten of interest (e.g., biotinylated 5-HIAA, melatonin or kynurenic acid). Patient serum or a dilution of the serum was admixed with the antibody against the metabolite (hapten) of interest (e.g., the anti-5-HIAA antibody), and then transferred to the plate. The plate was incubated for 1 hour at room temperature. The incubation condition was selected to provide sufficient time for the antibody to bind to the biotinylated hapten or to the metabolite in the serum. The plate was washed several times with wash buffer, e.g., PBS buffer. A secondary antibody, such as a goat anti-rabbit antibody-HRP conjugate or a goat anti-mouse antibody-HRP conjugate was added and the plate was incubated at room temperature for 1 hour. The plate was washed several times with wash buffer. A substrate solution was added for a detection reaction, e.g., color reaction, fluorescent reaction, chemiluminescent reaction, or luminescent reaction. The stop solution as added to arrest the substrate reaction. Then, the plate was read at an appropriate wavelength in a spectrophotometer to monitor the detection reaction. Based on the measured concentration of antibody bound to the biotinylated hapten, the concentration of the metabolite of interest can be calculated. In this type of assay, there is an inverse relationship between the amount of the metabolite in the sample and the measured level of bound antibody.

### Generation of Antibodies That Specifically Bind to 5-Hydroxyindole Acetic Acid (5-HIAA)

This example describes the generation of antibodies that specifically bind to a stable benzoxazole derivative of 5-hydroxyindole acetic acid (5-HIAA). The derivative includes a PEG linker and a carrier protein such as BSA. This example also shows that these antibodies can be used in immunoassays, such as a competitive ELISA to detect metabolites in samples, e.g., patient serum.

A. Synthesizing a Stable Benzoxazole Derivative of 5-HIAA containing a PEG Linker and a Carrier Protein or Biotin

The schemes below illustrate the synthesis of oxadole-indole intermediate 1, oxadole-indole intermediate 2, oxadole-indole intermediate 3, A-5, A-8, oxazolo-indole-PEG-SS-acid, oxazole-indole-PEG-biotin-ester, and oxadole-indole-PEG-biotin-acid.

### Step 1-Oxazolo-Indole Intermediate 1:

5-Hydroxy-1H-indo1-3-yl)-acetic acid (2.0 g, 10.46 mmol) was dissolved in anhydrous methanol (21 mL), anhydrous toluene (42 mL) was added to give a solution. 2M TMS-diazomethane in hexanes (5.2 mL, 10.46 mmol) was added while stirring at room temperature dropwise to give evolution of gas. Over a two hour period, two additional portions of 2M TMS-diazomethane in hexanes (2.6 mL, 5.23 mmol) were added at room temperature. Solvent was concentrated to a volume of 10 mL, toluene (20 mL) was added, solvent was concentrated to give an oil which was purified by SiO₂ flash chromatography using hexane/ethyl acetate, to give intermediate 1 as an oil (2.15 g, 95%). Step 1 is shown below.

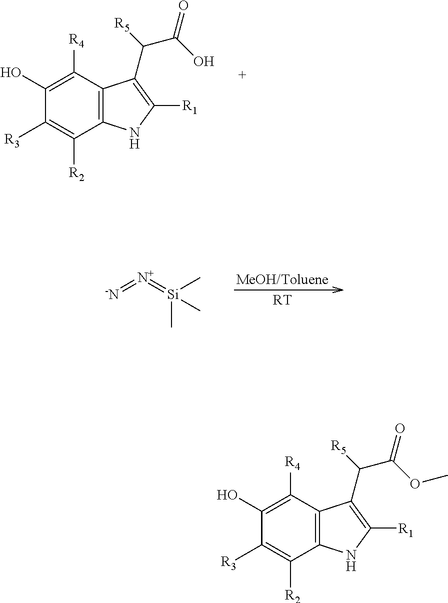

### Step 2-Oxazolo-Indole Intermediate 2:

Oxazolo-indole intermediate 1 (1000 mg, 4.87 mmol) was dissolved in anhydrous dimethoxyethane (DME) (92 mL), to give a solution which was cooled to 5° C. (4-Aminomethyl-benzyl)-carbamic acid tert-butyl ester (1.267 g, 5.36 mmol) was added, then MnO₂ (4.24 g, 48.7 mmol) was added to give a dark suspension which was allowed to warm to room temperature and stirred for 16 hours. The reaction mixture was cooled to 5° C., and additional MnO₂ (461 mg, 1.95 mmol) was added. The reaction mixture was allowed to warm to room temperature, stirred for 5 hours, then filtered through a celite pad (1 cm). The dark filtrate was concentrated, purified by SiO₂ flash chromatography using hexane/ethyl acetate to give intermediate 2 as a pale yellow solid (442 mg, 21%). Step 2 is shown below, wherein R₁-R₁₀=X, H, alkyl, acid group, aryl ester, alkyl ester or sulfonate and any combination of these groups.

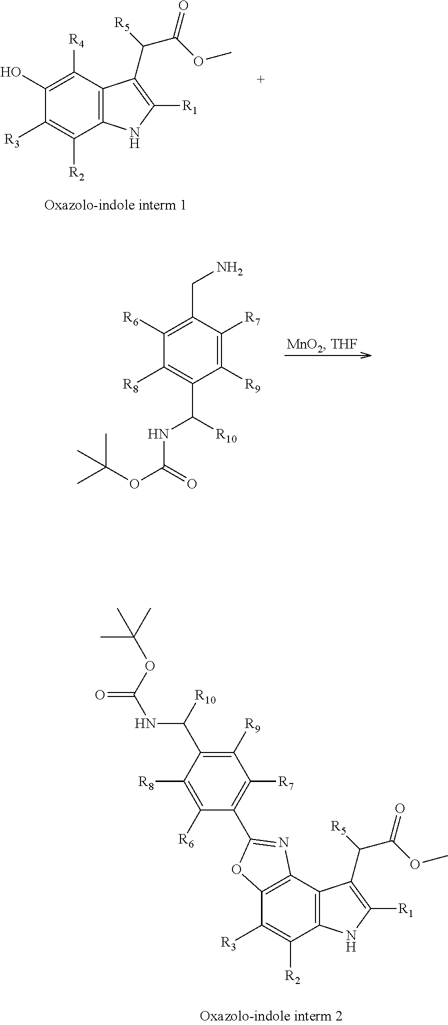

### Step 3-Oxazolo-Indole Intermediate 3:

Oxazolo-indole intermediate 2 (442 mg, 1.02 mmol) was suspended in DCM (4.0 mL), thioanisole (0.404 mL, 3.44 mmol), then TFA (1.9 mL, 24.6 mmol) was added dropwise at room temperature to give a solution. After 1.5 hour, the reaction mixture was diluted with toluene (20 mL) to give an oil, the solvent was concentrated to give a green suspension which was co-evaporated with toluene (20 mL) to a volume of 10 mL to give a suspension. The solids were filtered off, washed with toluene 5 times, then hexanes 5 times, to give a green solid which was dried in a vacuum oven (0.1 mm Hg) to give a non-hygroscopic green solid (557 mg, 50 wt % purity assumed). Step 3 is shown below.

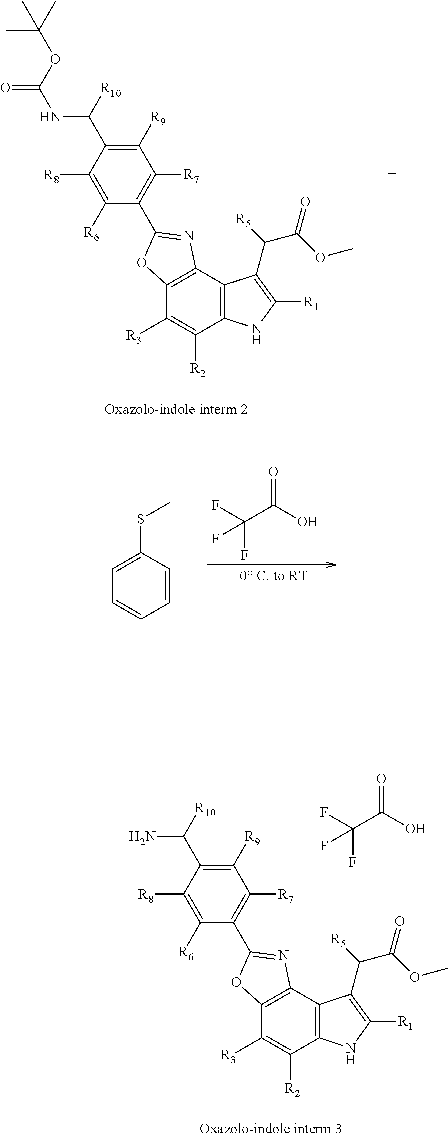

### Step 4-Oxazolo-Indole-PEG-SS-Acid:

Oxazolo-indole-PEG-SS-ester (68 mg, 0.0439 mmol) was dissolved in dioxane (1.4 mL) by gentle heating to give a solution which was cooled to RT. Aqueous 1.0 M LiOH (0.351 mL, 0.351 mmol) was added dropwise at RT while stirring to give a solution which was stirred at room temperature for 4 h. The solvent was concentrated to give an oil which was suspended in dioxane (1.4 mL), and the mixture was acidified with 1 N HCl (0.351 mL, 0.351 mmol) to pH 1 to give a solution. The solvent was concentrated to give a residue which was mostly dissolved in MeOH (25 mL). The mixture was filtered, and the filtrate was concentrated to give an oil which was purified by HPLC (CH₃CN—H₂O, 0.1% TFA) to give the title compound as an oil (20 mg, 30%).

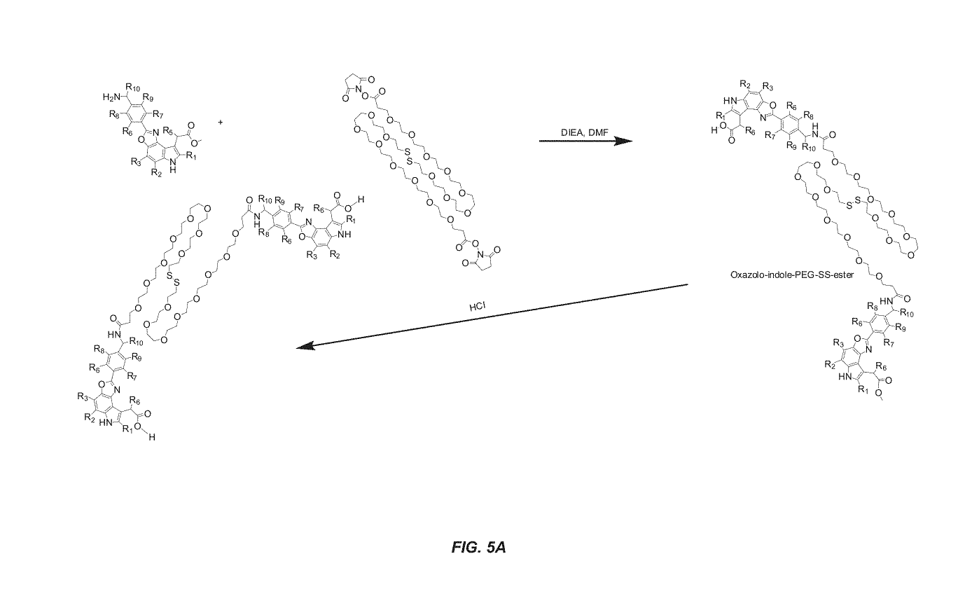

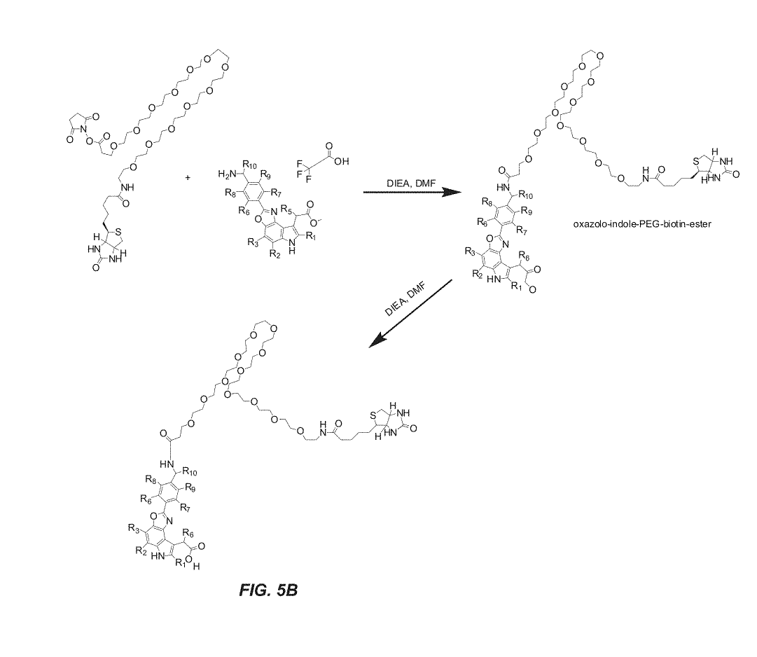

Oxazolo-indole-PEG-biotin-ester. This step describes the synthesis of an oxazolo-indole-PEG-biotin-ester derivative of 5HIAA. PEG-biotin-N-hydroxysuccinimide ester (100 mg, 0.106 mmol) was dissolved in anhydrous DMF (0.35 mL), oxazolo-indole interm 3 (95.5 mg, 0.212 mmol, 93% purity), then DIEA (0.148 mL, 0.850 mmol) were added at room temperature to give a solution which was stirred at RT for 2 days. The solvent was concentrated to give an oil (246 mg) which was purified by HPLC (CH₃CN—H₂O, 0.1% TFA) to give the title compound as an oil (123 mg, 100%). The step is shown in FIG. 5A.

Oxazolo-indole-PEG-biotin-acid. This step describes the synthesis of an oxazolo-indole-PEG-biotin-acid derivative of 5HIAA. Oxazolo-indole-PEG-biotin-ester (160 mg, 0.138 mmol) was dissolved in dioxane (2.2 mL) to give a solution. Aqueous 1.0 M LiOH (0.551 mL, 0.551 mmol) was added dropwise at room temperature while stirring to give a turbid solution which was stirred at room temperature for 6 hours. The solvent was concentrated to give a residue which was dissolved in H₂O (2.8 mL), and the mixture was acidified to pH 1 with 1 N HCl (0.414 mL, 0.411 mmol) at 4° C. to give a turbid solution. The solvent was concentrated in vacuo (1 mm Hg) at 30-40 C to give a residue (120 mg) which was purified by HPLC (CH₃CN—H₂O, 0.1% TFA) to give the title compound as an oil (61 mg, 50%). The step is shown in FIG. 5B.

### B. Generating Antibodies Against Benzoxazole Derivatives of 5-HIAA

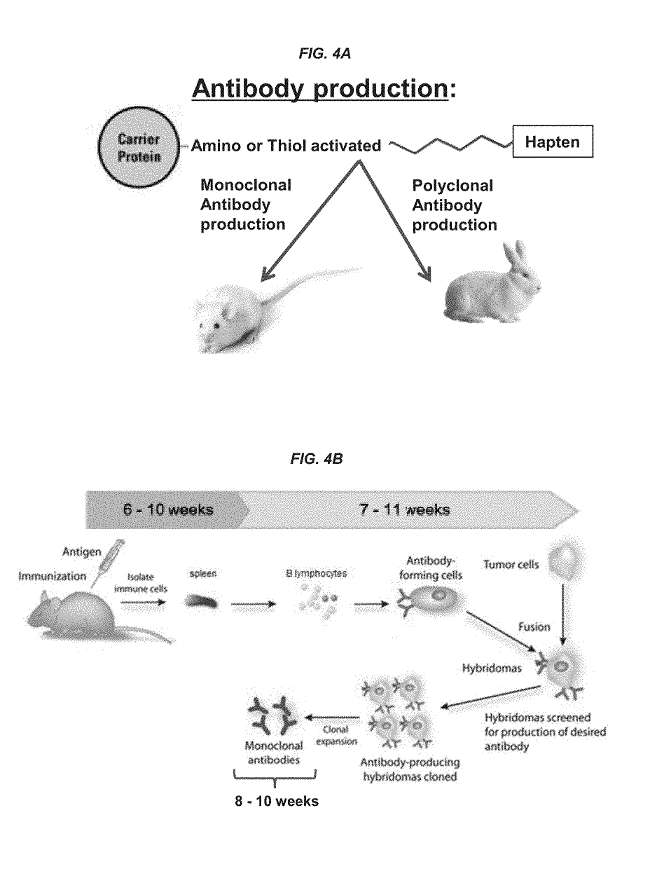

Monoclonal antibodies against the benzoxazole derivative of 5HIAA described herein were produced. For example, the oxazolo-indole-PEG-SS-acid was linked to a carrier protein via amine or thiol activation (FIG. 3A). The immunogen was injected into mice to generate monoclonal antibodies or rabbits to produce polyclonal antibodies (FIGS. 4A and 4B). Standard methods known to those skilled in the art were used for antibody generation, for example, techniques as described in ANTIBODIES, A LABORATORY MANUAL, Harlow and Lane, Eds., Cold Spring Harbor Laboratory, Cold Spring Harbor, N.Y. (1988).

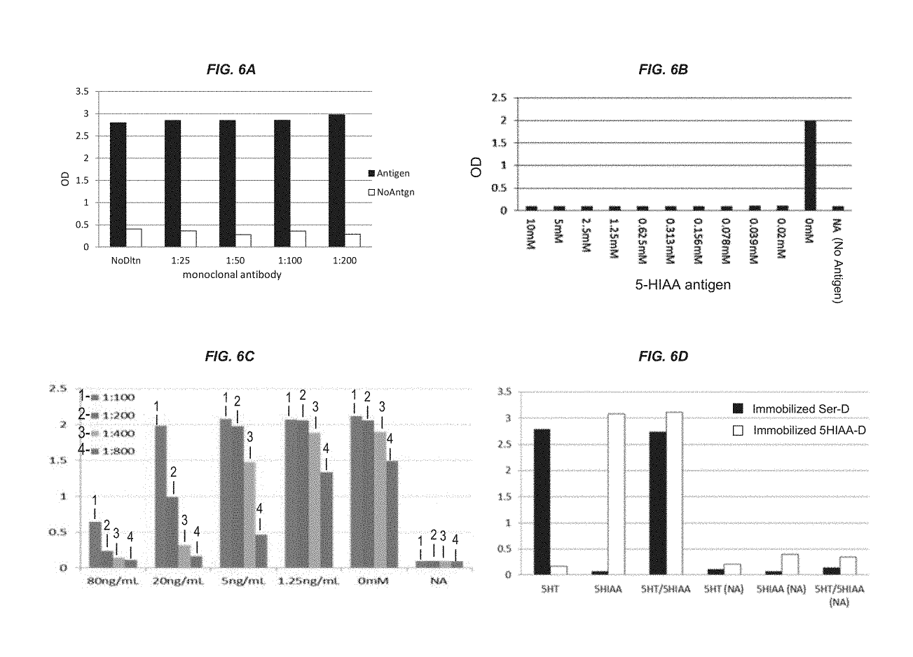

The benzoxazole derivative of 5HIAA was also linked to biotin instead of a carrier protein (FIG. 3A). Such a biotinylated hapten was used to test the validity and specificity of the antibodies generated using the following assay. The biotinylated hapten of interest (2 μg/ml) was coated onto a streptavidin plate for 1 hour at room temperature. The antigen was coated on the plate for about 2 hours at about 4° C. To evaluate the specificity or sensitivity of mouse monoclonal antibodies generated against the derivatized antigen, the antibodies were added to the wells and incubated for about 1 hour at room temperature. The plate was washed several times with wash buffer, e.g., PBS and the like. A goat anti-mouse antibody-HRP conjugate was added and incubated for about 1 hour at room temperature. The plate was washed several times with wash buffer. A color substrate was added for the colorimetric reaction. A stop solution was added prior to reading the plate at about 405 nm. FIG. 6A shows a titration experiment of the mouse monoclonal antibody. The titer of the antibody was very high at a 1:200 dilution and can be diluted further.

In the competitive ELISA assay described herein for 5-HIAA, free 5-HIAA competed with biotinylated 5-HIAA that was bound to the assay plate for the monoclonal antibody. FIG. 6B shows that increasing amounts of free 5-HIAA (0 ng/mL to 100 ng/mL; right to left) resulted in less monoclonal antibody bound to the biotinylated 5-HIAA (or a lower OD). FIG. 6C shows a titration of anti-5HIAA monoclonal antibody at different dilutions (1:100-1:800) at different concentrations of 5-HIAA (0 ng/mL to 80 mg/mL). FIG. 6D shows that the monoclonal antibody specific to 5-HIAA was not immunoreactive to derivatived serotonin (5-HT). The graph also shows that a monoclonal antibody against serotonin did not bind to derivatized 5-HIAA.

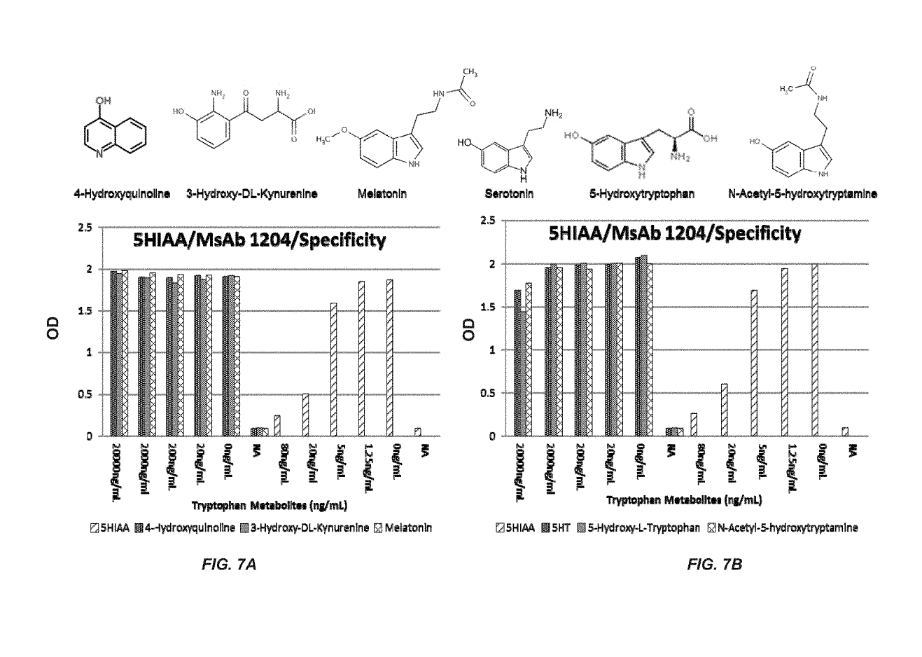

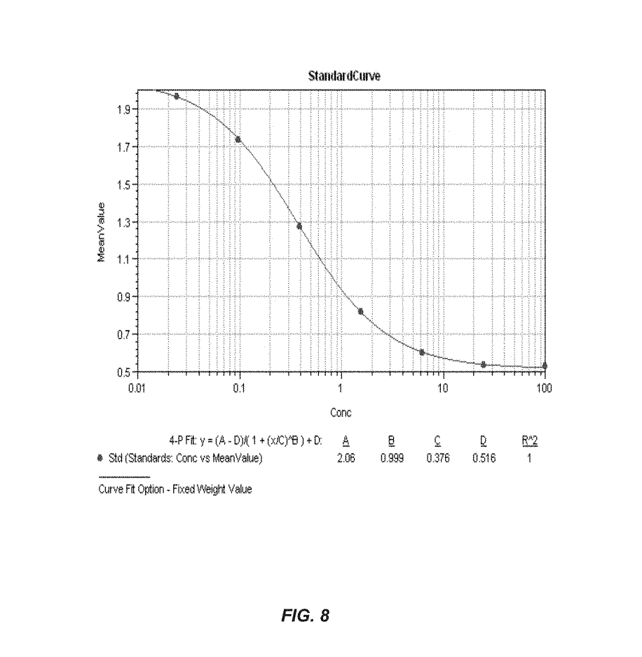

Assays of antibody specificity showed that the monoclonal antibodies against 5-HIAA were specific and do not bind to similar compounds, such as serotonin, melatonin, 5-hydroxy tryptophan, or tryptophan. In fact, these other compounds show 0 to <0.5% cross-reactivity to the monoclonal antibodies (FIGS. 7A and 7B). FIG. 8 shows that a standard curve can be generated with the monoclonal antibody against 5-HIAA in an immuno-based assay.

Generation of Antibodies that Specifically Bind to Melatonin

This example describes the generation of antibodies that specifically bind to melatonin. The example shows that the antibodies can be used in immunoassays such as competiton ELISA to detect melatonin in a patient sample.

Melatonin (5-methoxy-N-acetyltryptamine) is a compound derived from serotonin. Serotonin N-acetyltransferase converts serotonin to N-acetyloserotonin which is converted to melatonin by hydroxyindole-O-methyl transferase.

Melatonin may play a role in the pathogenesis of IBS (Konturek et al., J Physiol Pharmacol, 2007, 58:381-405; Bebeuik et al., J Pineal Res, 1994, 16:91-99). It manifests strong anti-oxidant and anti-inflammatory activity. It also regulates intestinal motility. Studies have shown that melatonin may have an inhibitory effect on smooth muscle motor activity. IBS has been associated with abnormal gastrointestinal motor functions, visceral hypersensitivity, psychosocial factors, autonomic dysfunction, and mucosal inflammation.

### A. Generating Immunogens Containing Melatonin

Melatonin was synthetically produced. A PEG (PEG₁-PEG₂₀) linker was attached (conjugated) to melatonin. A carrier protein such as BSA was then attached to the free end of the linker via amino or thiol activation (FIG. 3B). This melatonin antigen was used to produce polyclonal and monoclonal antibodies that specifically bind to melatonin.

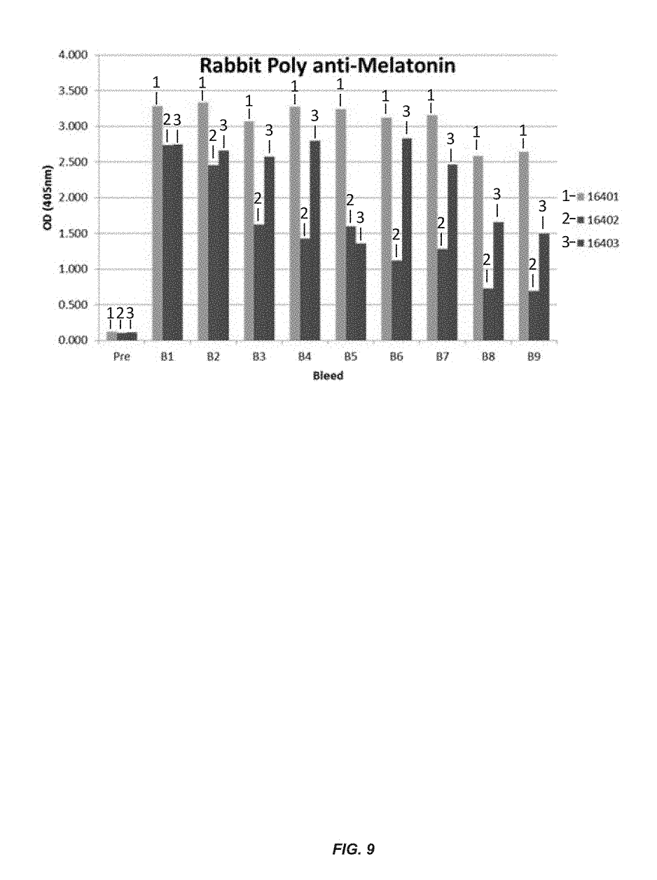

Antibodies were produced according to standard methods known to those skilled in the art. Monoclonal antibodies were generated according to methods such as those described in, e.g., Greenfield, EA. “Generating Monoclonal Antibodies” in ANTIBODIES: A LABORATORY MANUAL, 1^{st} edition, CSHL Press, New York, 1988. Polyclonal antibodies were raised by immunizing rabbits with the melatonin antigen and an adjuvant. The rabbits received booster immunizations of the melatonin antigen to increase their immune response and antibody titer. FIG. 9 shows that antibody titer from three immunized rabbits at pre-bleed, and bleeds 1-9. The graph shows that rabbit #16401 (1) produced antibodies that specifically bind to melatonin.

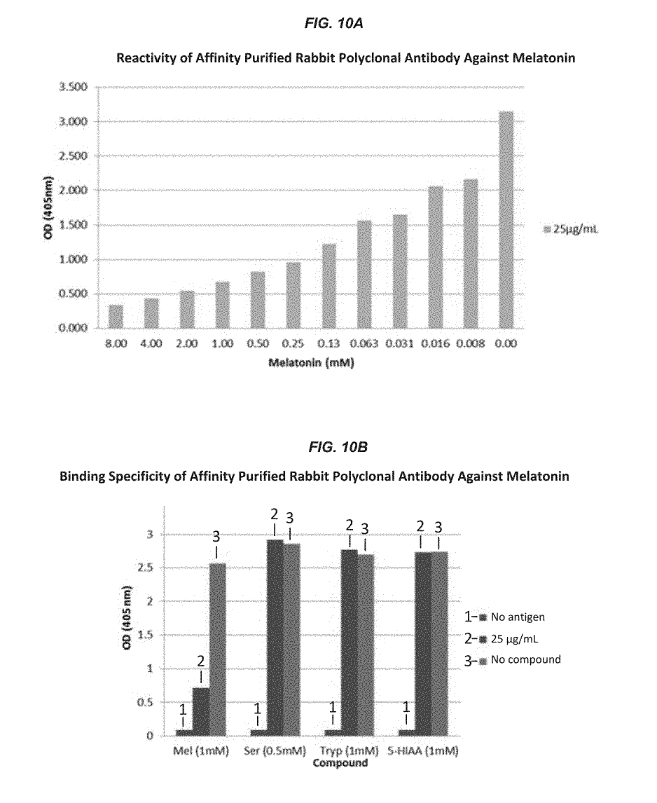

A biotinylated melatonin conjugate was also synthetically produced. A PEG (PEG₁-PEG₁₂) linker was attached (conjugated) to melatonin. Biotin was then attached to the free end of the linker via amino or thiol activation. This conjugate was used in an immunoassay to test the affinity and specificity of the anti-melatonin antibodies described herein (FIGS. 10A and 10B).

### B. Assaying Antibodies Against Melatonin

To test the validity and specificity of the anti-melatonin antibodies generated, the following assay was used. The biotinylated melatonin was coated onto a streptavidin plate for 1 hour at room temperature. The plate was washed and blocked with blocking buffer (e.g., SuperBlock™ buffer) to minimize non-specific binding. Rabbit antisera was serially diluted (1:100, 1:125; 1:250, 1:500, 1:1000) and transferred to individual wells of the plate. In a competitive immunoassay, a competing (test) compound was added to the wells and incubated for about 1 hour at room temperature. In some instances, the test compound was melatonin, or structurally similar compound such as serotonin, tryptophan, 5-HIAA, and the like. In some wells, no test compound was added.

The plate was incubated at room temperature (RT) of about 1 hour with orbital shaking. The plate was washed several times with wash buffer (e.g., PBST). Goat anti-rabbit antibody-horseradish peroxidase (HRP) conjugate was diluted (1:5000), added to each well, and incubated for 1 hour at RT. The plate was washed several times in wash buffer (e.g., PBST) to remove excess HRP conjugate. A color substrate was added and the plate was incubated at RT for the HRP-catalyzed reaction to generate a detectable color (e.g., 15 minutes in the dark). After color development, the stop solution (e.g., 4N NaOH) was added to stop the substrate reaction. The plate was read at about 405 nm or an appropriate wavelength for the detection reaction.

The assay was used to determine the binding activity and specificity of polyclonal antibodies described herein. The assay was also modified to test monoclonal antibodies raised against melatonin by using a goat anti-mouse antibody-HRP conjugate instead to a secondary antibody that recognizes rabbit antibodies.

To determine if the antibody against melatonin is specific to the antigen, a competing compound such as melatonin, serotonin, tryptophan and 5-HIAA was assayed. FIG. 10A shows that with a decreasing amount of melatonin (8.00 mM to 0 mM), more polyclonal antibody was detected as binding to the immobilized biotinylated melatonin. FIG. 10B shows that the addition of 1 mM melatonin in the competition assay decreased the amount of antibody bound to the biotinylated melatonin. In contrast, the addition of serotonin, tryptophan or 5-HIAA did not change the amount of anti-melatonin antibody bound to the immobilized antigen. The data shows that the anti-melatonin polyclonal antibody is highly specific for melatonin and has no cross-reactivity to compounds that are structurally similar to melatonin.

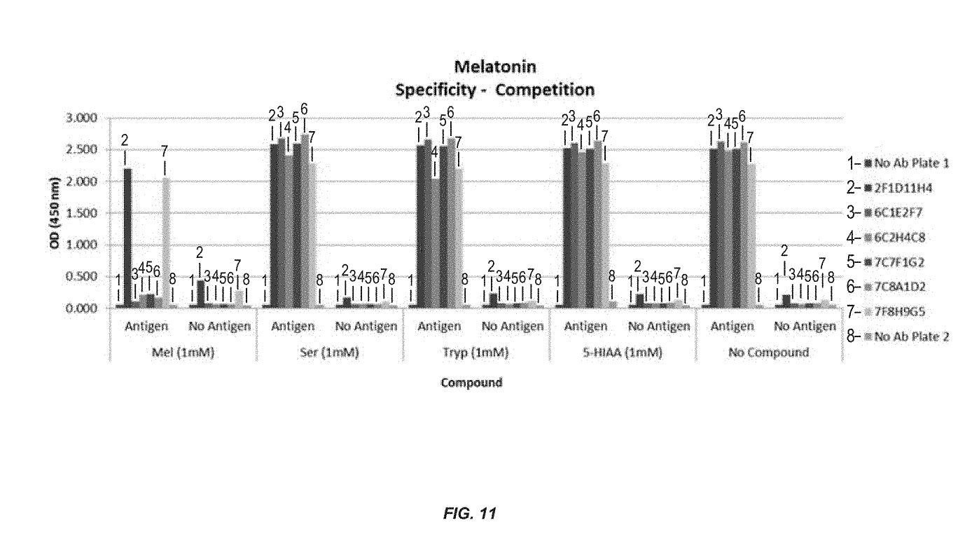

A similar competitive ELISA was performed to test the specificity of monoclonal antibodies from different hybridoma clones (2F1D11H4, 6C1E2F7, 6C2H4C8, 7C7F1G2, 7C8A1D2, and 7F8H9G5). The monoclonal antibodies were incubated with a test compound (1 mM melatonin, 1 mM serotonin, 1 mM tryptophan, or 1 mM 5-HIAA) prior to adding to the wells coated with immobilized melatonin. FIG. 11 shows that antibodies from clones 6C1E2F7, 6C2H4C8, 7C7F1G2, and 7C8A1D2 were specific for melatonin and did not bind to metabolites with a structure similar to melatonin.

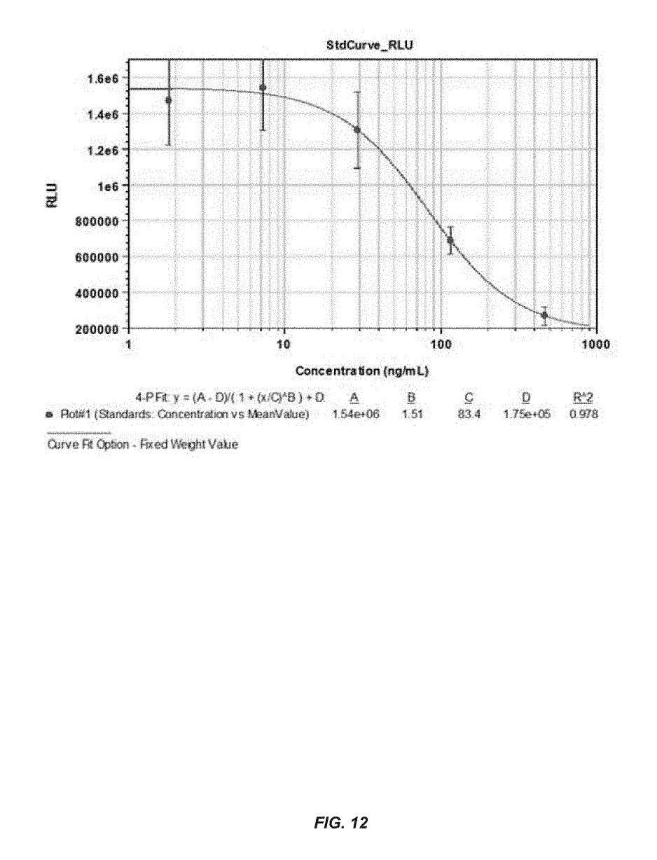

The sensitivity of the monoclonal anti-melatonin antibody was assayed using a standard ELISA. The biotinylated melatonin was immobilized onto a streptavidin coated multiwell plate. A serial dilution of the monoclonal antibody was added to the plate such that a standard curve could be generated. The plate was incubated for about 1 hour at room temperature. The wells were washed several times with a wash buffer. A HRP conjugated secondary antibody (goat anti-mouse IgG) was added and incubated for about 1 hour at room temperature. The plate was washed several times with a wash buffer. A colorimetric detection reagent was added. To stop the reaction, a stop reagent was added. The plate was read at the appropriate wavelength. FIG. 12 shows the standard curve for a monoclonal antibody that specifically binds to melatonin (from clone 6C1E2F7). The specificity for the antibody is 7.26 ng/ml.

### Generation of Antibodies That Specifically Bind to Kynurenic Acid (KYNA)

### A. Synthesis of Kynurenic Acid Immunogens for Making Polyclonal Antibodies

Compound 24: 6-(6-aminohexanamido)-4-hydroxyquinoline-2-carboxylic acid.

The scheme below illustrates the synthesis of Compound 24.

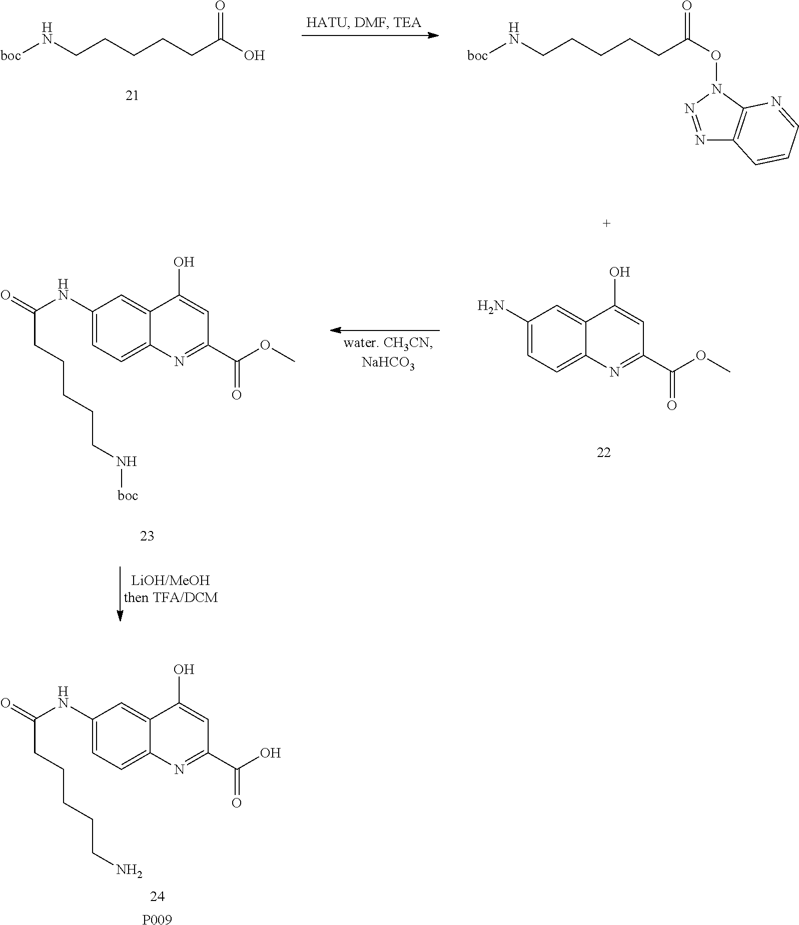

### Step 1:

A mixture of boc-amino-hexanoic acid (21, 277 mg, 1.2 mmol), DIPEA (0.21 ml, 1.2 mmol) and HATU (456 mg, 1.2 mmol) were stirred in DCM (5 ml) and acetonitrile (5 ml) for 30 min.

### Step 2:

To compound 22 (218 mg, 1 mmol) in a mixture of water (5 ml) and acetontrile (5 ml), NaHCO₃ (840 mg, 10 mmol) was added, followed by addition of reaction mixture from step 1 slowly with vigorously stirring. The mixture was stirred for additional 4 hrs afterwards, then acidified by sat. NaHSO₄, which resulted in precipitation. The solid was filtered to give rise to the intermediate 23.

### Step 3:

The solid 23 from step 2 was stirred with LiOH-H₂O (410 mg, 10 mmol) in MeOH (10 ml) at 60° C. for 4 hrs, then acidified to pH 3 by sat NaHSO₄ solution, concentrated. The resulting solid was filtered, washed with water, dried. It was then suspended in DCM (2 ml), followed by addition of TFA (2 ml). The slurry was stirred at room temperature for 4 hrs and then concentrated. Resulting solid was stirred with ethyl acetate (30 ml) for 5 min, insoluble was filtered and washed with ethyl acetate and dried to yield a grey solid as the desired compound 24 (120 mg). MS: 318.0 (M+H)⁺ 6-(6-aminohexanamido)-4-hydroxyquinoline-2-carboxylic acid.

To generate an immunogenic conjugate of KYNA, a PEG (PEG₁-PEG₂₀) linker was attached (conjugated) to chemically synthesized KYNA hapten and a carrier protein such as BSA was then attached to the free end of the linker via amino or thiol activation (FIG. 3C). The KYNA antigens described herein used to produce polyclonal antibodies that specifically bind to KYNA.

Synthesis of Compound 27: 4-hydroxy-6-(6-(6-(5-(3aS,4S,6aR)-2-oxohexahydro-1H-thieno[3,4-d]imidazol-4-yl)pentanamido)hexanamido)hexanamido)quinoline-2-carboxylic acid.

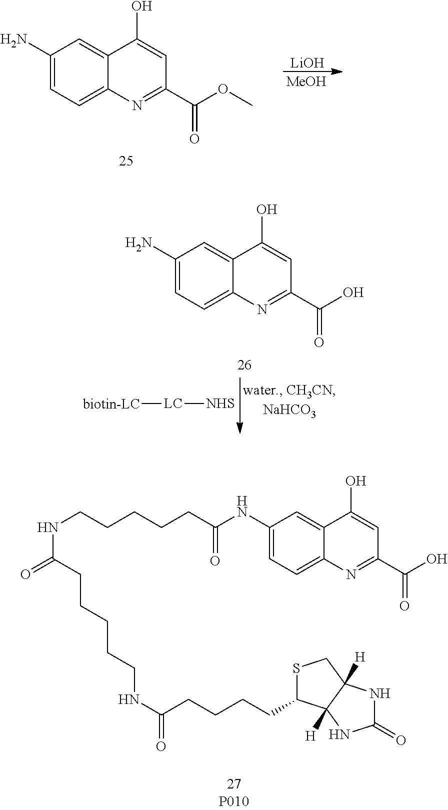

A mixture of compound 25 (327mg, 1.5 mmol) and LiOH—H₂O (430 mg, 10 mmol) were stirred in MeOH (10 ml) overnight, then carefully acidified 6N HCl to pH 7, concentrated to remove MeOH. The crude was then diluted with acetonitrile and water (10 ml/10 ml), and NaHCO₃ (1.26 g) was added, followed by addition of Biotin-LC-LC-NHS (852 mg, 1.5 mmol). The mixture was stirred vigorously for 1 day, acidified by 6N HCl, the resulting solids were filtered, and washed with MeOH, followed by water, then dried to produce the pure compound 27 (140 mg). MS: 657.2(M+H)⁺, 4-hydroxy-6-(6-(6-(5-((3aS,4S,6aR)-2-oxohexahydro-1H-thieno[3,4-d]imidazol-4-yl)pentanamido)hexanamido)hexanamido)quinoline-2-carboxylic acid.

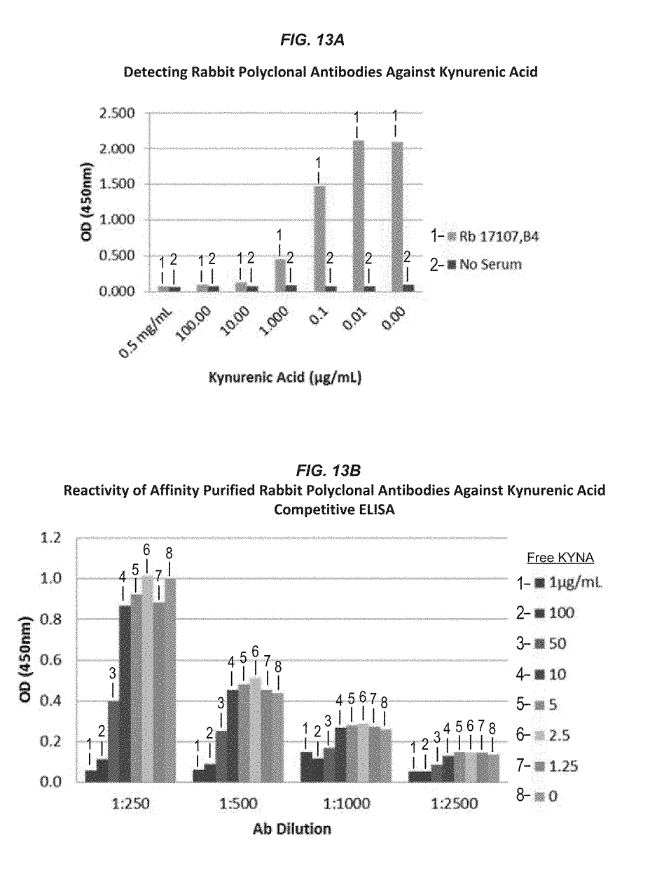

A biotinylated KYNA conjugate was also synthetically produced. A PEG (PEG₁-PEG₂₀) linker was attached (conjugated) to a KYNA hapten. Biotin was then attached to the free end of the linker via amino or thiol activation. This conjugate was used in immunoassays to test the affinity and specificity of the anti-KYNA antibodies described herein (FIGS. 13A, 13B, 14A, 14B, 15A, 16A and 16B).

### B. Synthesis of Kynurenic Acid Immunogens for Making Monoclonal Antibodies

Provided herein is a method of synthesizing kynurenic acid.

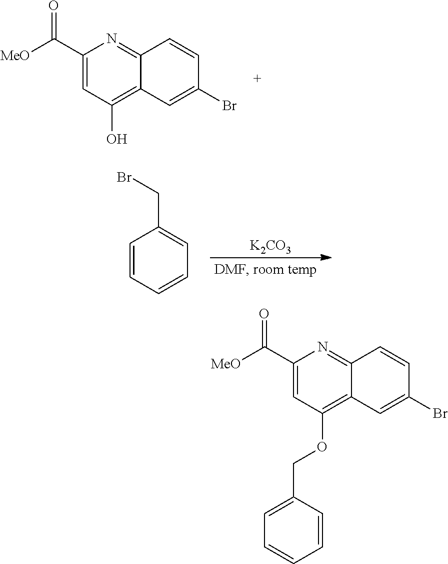

In a 100 mL round bottom flask, 6-Bromo-4-hydroxy-quinoline-2-carboxylic acid methyl ester (Kynurenic acid methyl ester) (564 mg, 2.0 mmol) was dissolved in dry DMF (12 mL), then added K₂CO₃ powder (691 mg, 5.0 mmol) to the reaction flask. After 5 min, benzyl bromide (0.285 mL, 2.4 mmol) was added using a syringe. The reaction continued at room temperature for 4 hours. The reaction was tested by TLC using 20% EtOAc in hexane, complete conversion to the product was observed. Then water (15 mL) was added to the reaction mixture and extracted with into EtOAc (3×20 mL). The organic layer was combined and washed with water (20 mL) and 1.0 N HCl (20 mL) and brine (20 mL). The organic layer was dried over sodium sulfate and evaporated. Then, product was purified by vacuum column chromatography (VCC) using 10-50% EtOAc-hexane. Pure product fractions were combined and evaporated to give the desired product 4-Benzyloxy-6-bromo-quinoline-2-carboxylic acid methyl ester (285 mg) as tan solid in pure state and 420 mg with some impurity. 1H NMR (499 MHz, Chloroform-d) δ8.43 (d, J=2.2 Hz, 1H), 8.10 (d, J=9.0 Hz, 1H), 7.83 (dd, J=9.0, 2.3 Hz, 1H), 7.70 (s, 1H), 7.57-7.50 (m, 2H), 7.49-7.38 (m, 3H), 5.37 (s, 2H), 4.08 (s, 3H).

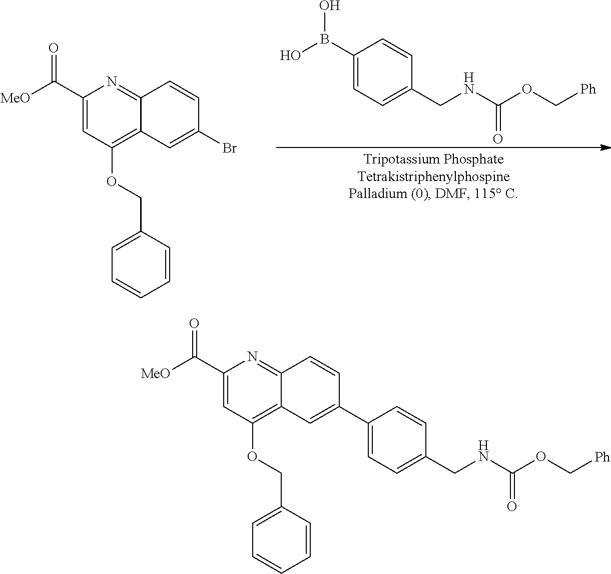 [SMILES: COC(=O)c1cc(OCc2ccccc2)c2cc(Br)ccc2n1 ; O=C(NCc1ccc(B(O)O)cc1)OCc1ccccc1 ; COC(=O)c1cc(OCc2ccccc2)c2cc(-c3ccc(CNC(=O)OCc4ccccc4)cc3)ccc2n1]

In a 25 mL round bottom flask, 4-Benzyloxy-6-bromo-quinoline-2-carboxylic acid methyl ester (372 mg, 1.0 mmol) was dissolved in DMF (5.0 mL) and degassed. Then tri potassium phosphate (467 mg, 2.2 mmol) and tetrakistriphenyl phosphine palladium (0) (57.75 mg, 0.05 mmol) added to the flask and continued heating for 16 hours at 115° C. After completion, evaporated volatiles and added water (5.0 mL) and extracted with EtOAc (3×20 mL). Combined organic layer dried over sodium sulfate and evaporated. Then, purified on vacuum column chromatography using hexane-EtOAc (0-100%). Desired product eluted at 50% EtOAc. Pure product fractions combined and evaporated to give 4-Benzyloxy-6-[4-(benzyloxycarbonylamino-methyl)-phenyl]-quinoline-2-carboxylic acid methyl ester (255 mg, 48% yield) as a light green yellow solid confirmed by LCMS and NMR. ¹H NMR (499 MHz, DMSO-d6) δ8.38 (d, J=1.8 Hz, 1H), 8.22-8.11 (m, 2H), 7.88 (t, J=6.1 Hz, 1H), 7.76 (d, J=7.9 Hz, 2H), 7.70 (s, 1H), 7.65-7.58 (m, 4H), 7.58-7.51 (m, 1H), 7.49-7.26 (m, 6H), 5.55 (s, 2H), 5.06 (s, 2H), 4.27 (d, J =6.3 Hz, 2H), 3.96 (s, 3H). MS: 533.5 [M+H] calculated for C₃₃H₂₈N₂O₅.

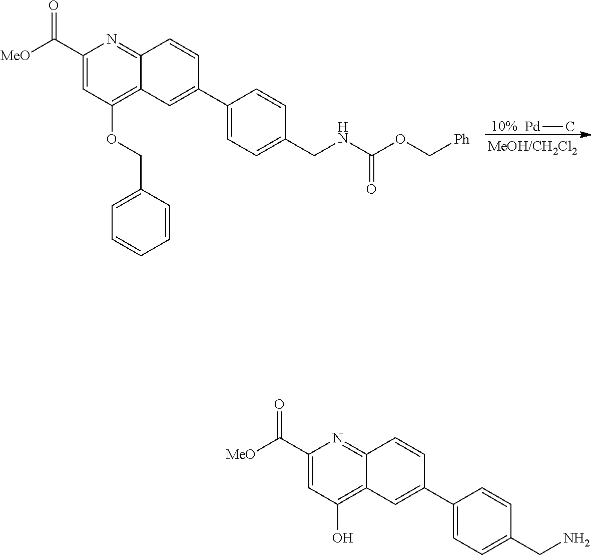

Provided herein is a method of synthesizing biotinylated kynurenic acid.

In a 250 mL round bottom flask, 4-Benzyloxy-6-[4-(benzyloxycarbonylamino-methyl)-phenyl]-quinoline-2-carboxylic acid methyl ester (532 mg, 1.0 mmol) was dissolved in MeOH (40 mL) and CH₂Cl₂ (30 mL). Then the solution was degassed and 10% Pd—C (85 mg) added. Then the solution was hydrogenated using balloon for overnight, then analyzed on LC-MS and TLC. The reaction solution was filtered through a celite bed and washed the celite layer with methanol. Then the clear solution obtained was acidified using concentrated HCl to visualize a yellow precipitate product. Then, the solution was evaporated to obtain yellow solid. LC MS: 309 [M+H] calculated for C₁₈H₁₆N₂O₃.

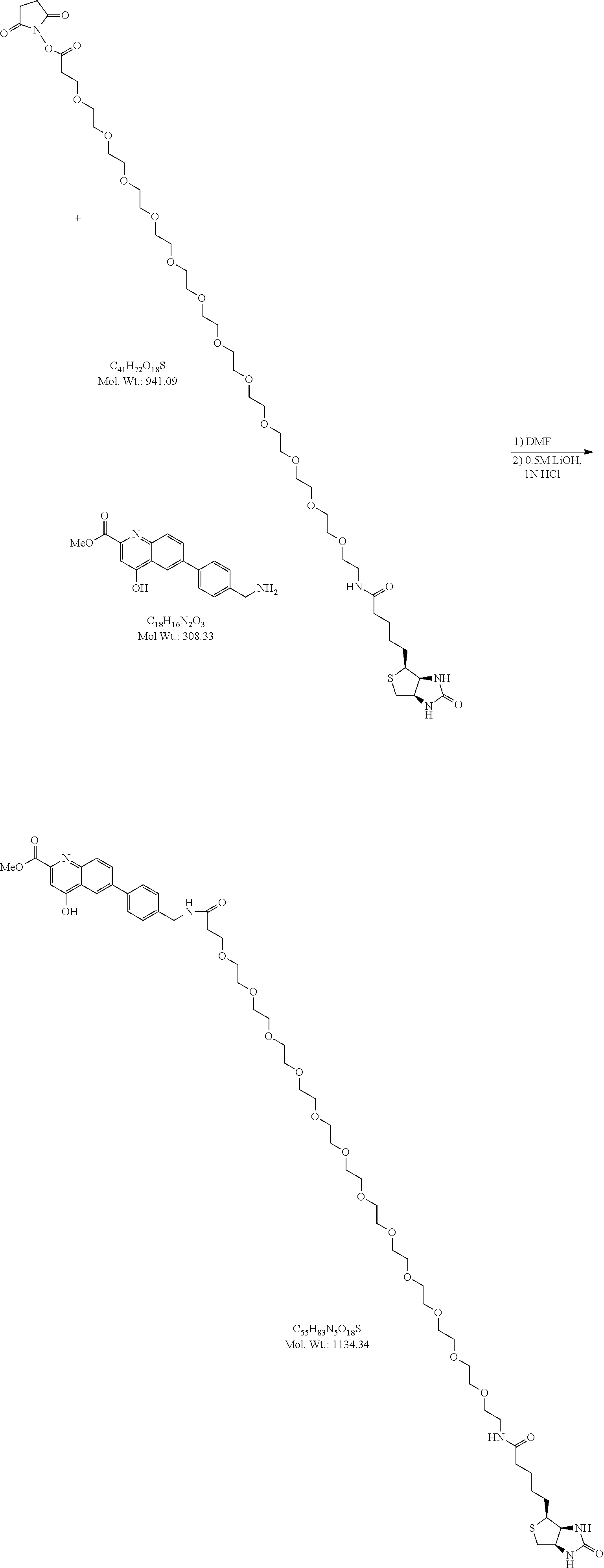 [SMILES: O=C(CCCC[C@@H]1SC[C@@H]2NC(=O)N[C@@H]21)NCCOCCOCCOCCOCCOCCOCCOCCOCCOCCOCCOCCOCCC(=O)ON1C(=O)CCC1=O ; COC(=O)c1cc(O)c2cc(-c3ccc(CN)cc3)ccc2n1 ; COC(=O)c1cc(O)c2cc(-c3ccc(CNC(=O)CCOCCOCCOCCOCCOCCOCCOCCOCCOCCOCCOCCOCCNC(=O)CCCC[C@@H]4SC[C@@H]5NC(=O)N[C@@H]54)cc3)ccc2n1]

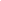

Provided herein is a method of synthesizing kynurenic acid-PEG-disulfide.

In a 4.0 mL brown glass vial, kynurenic amine hydrochloride (68 mg, 0.2 mmol) and PEG₁₂-Biotin-NHS ester (94.1 mg, 0.1 mmol) were suspended in DMF (1.0 mL) and stirred at room temperature for 16 hours. The presence of the product in the reaction mixture was confirmed using LCMS. The product was purified on silica gel vacuum column chromatography, eluting with CH₂Cl₂-MeOH (0-20%) as a gradient. Pure product fractions were combined and evaporated to yield a light yellow solid (42 mg). ¹H NMR (499 MHz, Methanol-d₄) δ8.49 (d, J=2.2 Hz, 1H), 8.10 (dd, J=8.8, 2.2 Hz, 1H), 7.96 (t, J=9.4 Hz, 2H), 7.78-7.67 (m, 2H), 7.45 (d, J=7.9 Hz, 2H), 6.97 (s, 1H), 4.57 (s, 1H), 4.47 (d, J=4.3 Hz, 3H), 4.29 (dd, J=7.9, 4.4 Hz, 1H), 4.06 (s, 3H), 3.79 (t, J=5.9 Hz, 2H), 3.70-3.45 (m, 54H), 3.35 (t, J=5.4 Hz, 2H), 3.24-3.12 (m, 1H), 2.92 (dd, J=12.7, 5.0 Hz, 1H), 2.68 (d, J=4.4 Hz, 1H), 2.53 (t, J=6.0 Hz, 2H), 2.21 (t, J=7.3 Hz, 2H), 1.79-1.53 (m, 4H), 1.44 (q, J=7.6 Hz, 2H). MS: 1133.3 [M-H] calculated for C₅₅H₃₃N₅O₁₈S.

Hydrolysis of methyl ester: The above obtained product was dissolved in THF (1.5 mL) and 0.5 M LiOH solution (0.4 mL) was added. The reaction was continued at room temperature for 2 hours, then was acidified with 1N HCl (0.3 mL). The sample was tested on LCMS. The desired product with sufficient purity (>85%) was observed. The sample was dried completely by connecting the sample to high vacuum overnight.

In a 4.0 mL brown glass vial, kynurenic amine hydrochloride (68 mg, 0.2 mmol) and SS-PEG-NHS ester (111 mg, 0.1 mmol) were suspended in DMF (1.0 mL) and stirred at room temperature for 16 hours. The presence of the product in the reaction mixture was confirmed the product using LCMS. The product was purified on silica gel vacuum column chromatography, eluting with CH₂Cl₂-MeOH (0-15%) as a gradient. Pure product fractions were combined and evaporated to yield a gummy solid (28 mg). ¹H NMR (499 MHz, Methanol-d4) δ8.48 (dd, J=21.2, 2.2 Hz, 1H), 8.13-8.02 (m, 1H), 7.93 (dd, J=19.9, 8.8 Hz, 1H), 7.71 (dd, J=16.0, 8.2 Hz, 2H), 7.44 (t, J=9.4 Hz, 2H), 6.96 (d, J=16.4 Hz, 1H), 4.54 (s, 3H), 4.46 (d, J=5.7 Hz, 2H), 4.05 (d, J=7.3 Hz, 4H), 3.83-3.66 (m, 8H), 3.66-3.47 (m, 50H), 3.23 (q, J=7.4 Hz, 2H), 2.92-2.82 (m, 3H), 2.67 (s, 4H), 2.53 (t, J=5.9 Hz, 2H), 1.37 (d, J=6.6 Hz, 17H). MS: 1494.6 [M-H] calculated for C₇₄H₁₀₂N₄O₂₄S₂.

Hydrolysis of methyl ester: The above obtained product was dissolved in THF (1.5 mL) and 0.5 M LiOH solution (0.4 mL) was added. The reaction continued at room temperature for 2 hours, and then was acidified with 1N HCl (0.3 mL). An aliquot of the sample was tested on LCMS. The desired product with sufficient purity (>85%) was observed. The sample was dried completely by connecting the sample to high vacuum overnight.

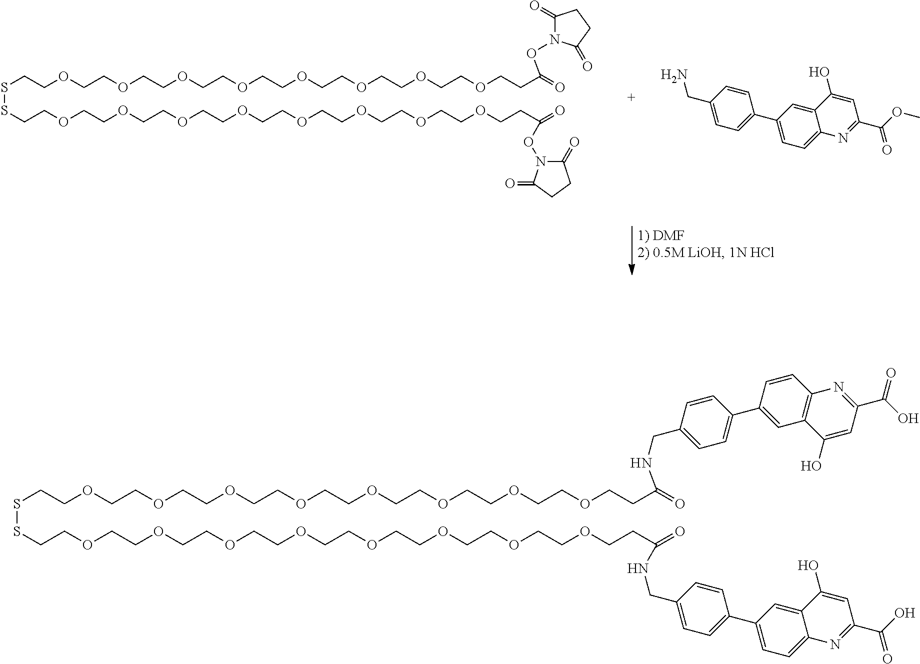 [SMILES: O=C(CCOCCOCCOCCOCCOCCOCCOCCOCCSSCCOCCOCCOCCOCCOCCOCCOCCOCCC(=O)ON1C(=O)CCC1=O)ON1C(=O)CCC1=O ; COC(=O)c1cc(O)c2cc(-c3ccc(CN)cc3)ccc2n1 ; O=C(CCOCCOCCOCCOCCOCCOCCOCCOCCSSCCOCCOCCOCCOCCOCCOCCOCCOCCC(=O)NCc1ccc(-c2ccc3nc(C(=O)O)cc(O)c3c2)cc1)NCc1ccc(-c2ccc3nc(C(=O)O)cc(O)c3c2)cc1]

### C. Antibodies Against Kynurenic Acid

Antibodies were produced according to standard methods known to those skilled in the art. Monoclonal antibodies were generated according to methods such as those described in, e.g., Greenfield, EA. “Generating Monoclonal Antibodies” in ANTIBODIES: A LABORATORY MANUAL, 1^{st} edition, CSHL Press, New York, 1988. Polyclonal antibodies were raised by immunizing rabbits with the melatonin antigen and an adjuvant. The rabbits received booster immunizations of the melatonin antigen to increase their immune response and antibody titer.

To test the validity and specificity of the anti-melatonin antibodies generated, the following assay was used. The biotinylated kynurenic acid was coated onto a streptavidin plate for 1 hour at room temperature. The plate was washed and blocked with blocking buffer (e.g., SuperBlock™ buffer) to minimize non-specific binding. Rabbit antisera was serially diluted and transferred to individual wells of the plate. In a competitive immunoassay, a competing (test) compound was added to the wells and incubated for about 1 hour at room temperature. In some instances, the test compound was melatonin, or structurally similar compound such as serotonin, tryptophan, 5-HIAA, kynurenine and the like. In some wells, no test compound was added.

The plate was incubated at room temperature (RT) of about 1 hour with orbital shaking. The plate was washed several times with wash buffer (e.g., PBST). Goat anti-rabbit antibody-horseradish peroxidase (HRP) conjugate was diluted (1:5000), added to each well, and incubated for 1 hour at RT. The plate was washed several times in wash buffer (e.g., PBST) to remove excess HRP conjugate. A color substrate was added and the plate was incubated at RT for the HRP-catalyzed reaction to generate a detectable color (e.g., 15 minutes in the dark). After color development, the stop solution (e.g., 4N NaOH) was added to stop the substrate reaction. The plate was read at about 405-450 nm or an appropriate wavelength for the detection reaction.

FIG. 13A shows that the reactivity of polyclonal antibodies against KYNA in serum from a rabbit immunized with a KYNA immunogen. The graph shows the results from a competitive ELISA assay where biotinylated KYNA was coated onto the surface of a multiwell plate. FIG. 13B shows that binding sensitivity of affinity purified rabbit anti-KYNA polyclonal antibodies. The antibodies were purified using a standard method. In the competitive ELISA the amount of polyclonal antibody was diluted form 1:250-1:2500 and different concentrations of free KYNA was evaluated.

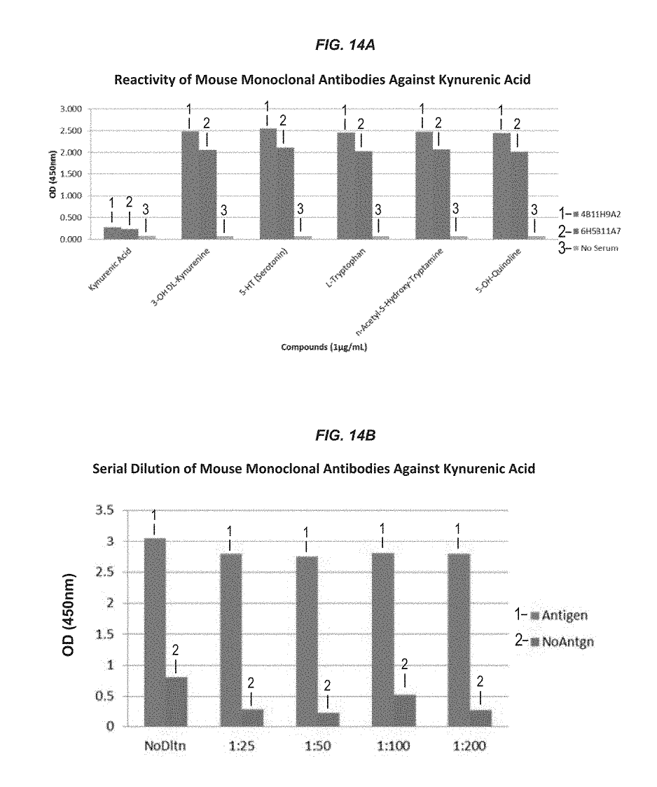

A similar competitive ELISA assay was used evaluate the specificity and sensitivity of the monoclonal antibodies generated as described herein. The results shows that antibodies from hybridoma clones 4B11H9A2 and 6H5B11A7 specifically bind to KYNA and have no cross-reactivity to 3-OH-DL-kynurenine, serotonin, tryptophan, n-acetyl-5-hydroxy-tryptamine, and 5-OH-quinoline (FIG. 14A). Compounds that are structurally similar to KYNA did not interfere with the binding of the antibody to KYNA. FIG. 14B shows a titration of the mouse monoclonal anti-KYNA antibody. The antibody remains immunoreactive to KYNA even when diluted.

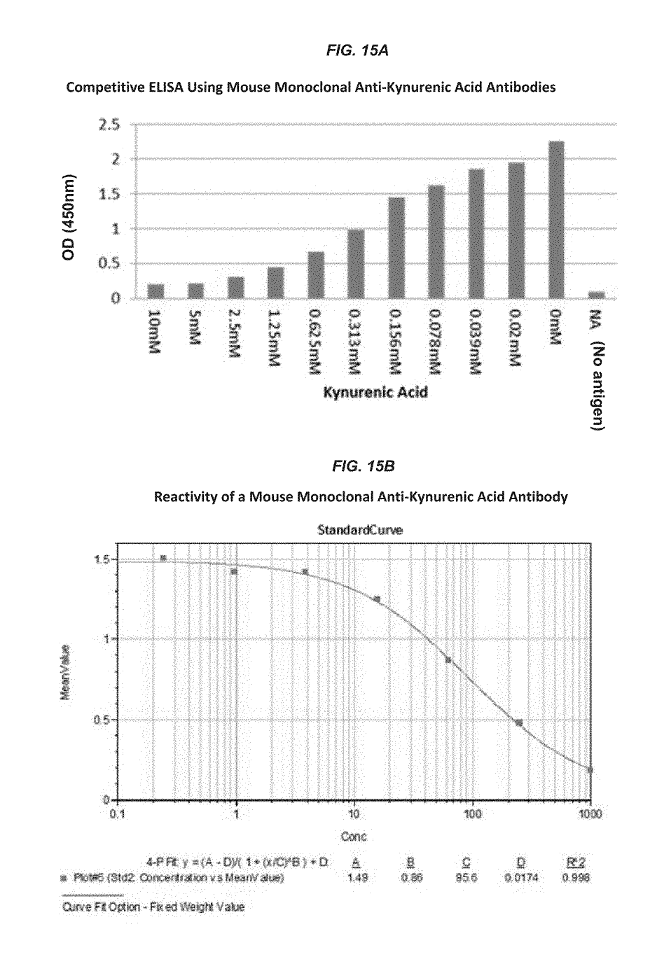

FIGS. 15A and 15B show that the mouse monoclonal antibodies produced by hybridoma clone 6H5B11A7 specifically bind to kynurenic acid. As shown in FIG. 15A, free KYNA antigen competes with immobilized KYNA antigen for antibody binding in the competitive ELISA provided herein. With increasing amounts of free KYNA, less antibody binds to the immobilized antigen and the OD value decreases. FIG. 15B shows a standard curve for the mouse monoclonal anti-KYNA antibody.

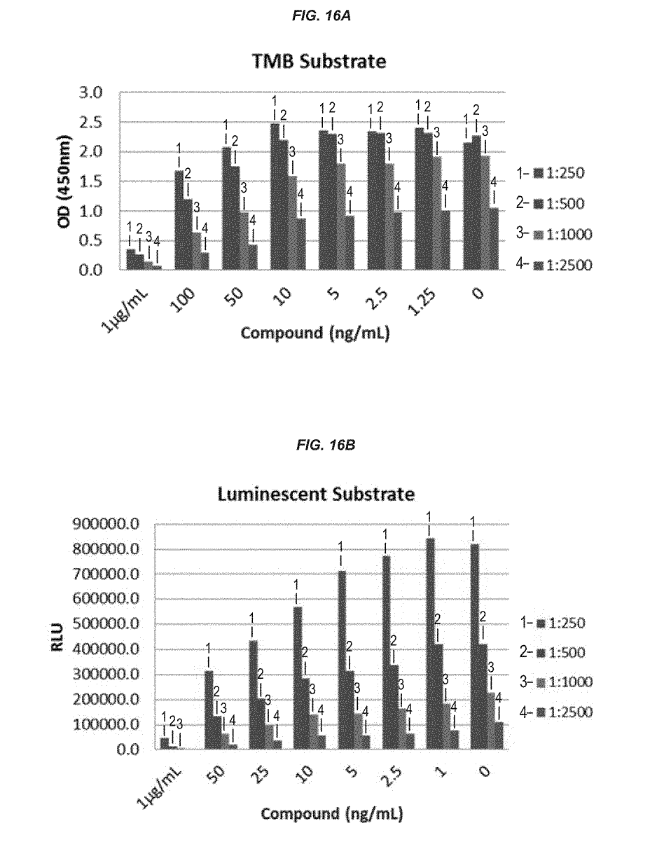

FIGS. 16A and 16B shows the results from an exemplary embodiment of the competitive ELISA disclosed herein. In FIG. 16A, a TMB substrate was used for the colorimetric reaction. In FIG. 16B, a luminescent substrate was used for the detection reaction. The assay using the luminescent substrate provides more sensitivity than the TMB substrate assay.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. An isolated antibody or antibody fragment thereof that specifically binds to 5-hydroxyindole-3-acetic acid (5-HIAA) and has less than 1% cross-reactivity to one or more members selected from the group consisting of tryptophan (Trp), serotonin (5-HT), 5-hydroxytryptophan (5-HTP), kynurenine (KYN), kynurenic acid (KYNA), 3-hydroxykynurenine (3-HK), 3-hydroxyanthranilic acid (3-HAA), quinolinic acid (QUIN), anthranilic acid (ANA), serotonin-O-sulfate, serotonin-O-phosphate, and melatonin (MT).

2. The isolated antibody or antibody fragment thereof of claim 1, wherein the antibody is a polyclonal antibody or a monoclonal antibody.

3. The isolated antibody or antibody fragment thereof of claim 1, wherein the antibody is a chimeric or a humanized antibody.

4. The isolated antibody or antibody fragment thereof of claim 1, wherein the antibody fragment is a Fab fragment, a Fab′ fragment or F(ab)′₂ fragment.

5. The isolated antibody or antibody fragment thereof of claim 1, wherein the antibody or antibody fragment thereof is produced by the hybridoma cell line deposited under ATCC Accession No. PTA-1222671 on Nov. 17, 2015 and designated 1204-10G6F11H3.

6. The isolated antibody or antibody fragment thereof of claim 1, wherein the antibody or antibody fragment thereof is produced by immunizing an animal with an immunogen comprising a 5-HIAA derivative conjugated to a carrier protein under conditions such that immune cells of the animal produce an antibody or antibody fragment thereof that specifically binds to 5-HIAA; and isolating the antibody or antibody fragment thereof from the animal.

7. The isolated antibody or antibody fragment thereof of claim 6, wherein the animal is a goat, rabbit or mouse.

8. The isolated antibody or antibody fragment thereof of claim 6, wherein the 5-HIAA derivative comprises a benzoxazole derivative of 5-HIAA.

9. An isolated antibody or antibody fragment thereof that specifically binds to melatonin (MT) and has less than 1% cross-reactivity to one or more members selected from the group consisting of tryptophan (Trp), serotonin (5-HT), 5-hydroxytryptophan (5-HTP), 5-hydroxyindole-3-acetic acid (5-HIAA), kynurenine (KYN), kynurenic acid (KYNA), 3-hydroxykynurenine (3-HK), 3-hydroxyanthranilic acid (3-HAA), quinolinic acid (QUIN), anthranilic acid (ANA), serotonin-O-sulfate, and serotonin-O-phosphate.

10. The isolated antibody or antibody fragment thereof of claim 9, wherein the antibody is a polyclonal antibody or a monoclonal antibody.

11. The isolated antibody or antibody fragment thereof of claim 9, wherein the antibody is a chimeric or a humanized antibody.

12. The isolated antibody or antibody fragment thereof of claim 9, wherein the antibody fragment is a Fab fragment, a Fab′ fragment or F(ab)′₂ fragment.

13. The isolated antibody or antibody fragment thereof of claim 9, wherein the antibody or antibody fragment thereof is produced by the hybridoma cell line deposited under ATCC Accession No. PTA-122669 on Nov. 17, 2015 and designated 1212-6C1E2F7.

14. The isolated antibody or antibody fragment thereof of claim 9, wherein the antibody or antibody fragment thereof is produced by immunizing an animal with an immunogen comprising melatonin conjugated to a carrier protein under conditions such that immune cells of the animal produce an antibody or antibody fragment thereof that specifically binds to melatonin; and isolating the antibody or antibody fragment thereof from the animal.

15. An isolated antibody or antibody fragment thereof that specifically binds to kynurenic acid (KYNA), which has less than 1% cross-reactivity to one or more members selected from the group consisting of tryptophan (Trp), serotonin (5-HT), 5-hydroxytryptophan (5-HTP), 5-hydroxyindole-3-acetic acid (5-HIAA), kynurenine (KYN), 3-hydroxykynurenine (3-HK), 3-hydroxyanthranilic acid (3-HAA), quinolinic acid (QUIN), anthranilic acid (ANA), serotonin-O-sulfate, serotonin-O-phosphate, and melatonin.

16. The isolated antibody or antibody fragment thereof of claim 15, wherein the antibody is a polyclonal antibody or a monoclonal antibody.

17. The isolated antibody or antibody fragment thereof of claim 15, wherein the antibody is a chimeric or a humanized antibody.

18. The isolated antibody or antibody fragment thereof of claim 15, wherein the antibody fragment is a Fab fragment, a Fab′ fragment or F(ab)′₂ fragment.

19. The isolated antibody or antibody fragment thereof of claim 15, wherein the antibody or antibody fragment thereof is produced by the hybridoma cell line deposited under ATCC Accession No. PTA-122670, on Nov. 17, 2015 and designated 1194-6H5B11A7.

20. The isolated antibody or antibody fragment thereof of claim 15, wherein the antibody or antibody fragment thereof is produced by immunizing an animal with an immunogen comprising kynurenic acid (KYNA) conjugated to a carrier protein under conditions such that immune cells of the animal produce an antibody or antibody fragment thereof that specifically binds to KYNA; and isolating the antibody or antibody fragment thereof from the animal.
